(19) United States
(12) Patent Application Publication
(10) Pub. No.: US 2024/0105292 A1
(43) Pub. Date: Mar. 28, 2024

(54) PLATFORM FOR SYNTHESIZING HIGH-DIMENSIONAL LONGITUDINAL ELECTRONIC HEALTH RECORDS USING A DEEP LEARNING LANGUAGE MODEL

(71) Applicant: MediSyn, Inc., Las Vegas, NV (US)

(72) Inventors: Brandon Philip Theodorou, Chicago, IL (US); Jimeng Sun, Las Vegas, NV (US)

(21) Appl. No.: 18/457,759

(22) Filed: Aug. 29, 2023

Related U.S. Application Data
(60) Provisional application No. 63/358,506, filed on Jul. 5, 2022.

(51) Int. Cl. G16H 10/60

(52) U.S. Cl. CPC G16H 10/60

(57) ABSTRACT

In one aspect, the present disclosure relates to a platform for creating synthetic electronic health records, the platform being configured to perform operations including receiving EHR data and encoding the received EHR data as a plurality of fixed length vectors to form a fixed-length matrix. The platform provides the fixed-length matrix to a machine learning model as input to produce a plurality of visit history representations. For one or more particular visit history representations, of the plurality of visit history representations, the platform applies code information associated with the particular visit history. One or more appended visit histories are provided to one or more masked linear layers to produce a probability matrix comprising probabilities for each code for each visit. The platform produces one or more synthetic EHRs based on repeated sequential generation of and sampling from the probability matrix.

### RELATED APPLICATION

Under provisions of 35 U.S.C. § 119(e), Applicant claims the benefit of U.S. Provisional Application No. 63/358,506 filed on Jul. 5, 2022, which is incorporated herein by reference.

It is intended that each of the referenced applications may be applicable to the concepts and embodiments disclosed herein, even if such concepts and embodiments are disclosed in the referenced applications with different limitations and configurations and described using different examples and terminology.

### FIELD OF DISCLOSURE

The present disclosure generally relates to generation of Electronic Health Record (EHR) Data, and particularly for synthesizing EHR data for use in developing machine learning solutions within the healthcare domain.

### BACKGROUND

With the recent movement in the past decade towards the standardization and digitization of electronic health records (EHRs) as well as the rapid improvement of machine learning techniques, a huge of amount of opportunity and progress has arisen in the intersection of medicine and artificial intelligence towards providing data and model-based solutions to health challenges. The applications and developments have ranged from health monitoring to computational phenotyping to treatment recommendation. However, such progress is not only constrained by the development of machine learning approaches and algorithms, but also by the other key aforementioned ingredient, EHR data. As such, access to large amounts of strong and well-formatted health record data is important to the progression of new healthcare solutions.

Unfortunately, despite the creation and widespread usage of various systems to collect and format health data at both the patient and visit level, much of the data available today suffers from two key limitations.

The first limitation is that of privacy. EHRs contain, by their nature, extremely sensitive information relating to the patients associated with the records. Consequently, the EHR (and the data therein) cannot be freely shared. Instead, the EHR data must be either de-identified, which often limits the utility of the data, or barriers to access must be put in place, preventing a user from accessing at least portions of the data in the EHR without authorization from the patient. This prevents open development, collaboration, and competition which have been important factors in the development of the field of machine learning as a whole, and thus holds back the development of machine learning for further possible healthcare solutions.

The second limitation is that, despite the huge amounts of EHR data which have been collected and standardized into usable datasets, there is nonetheless a problem of scarcity in both labeled data as a whole as well as specifically data of scarce labels, such as data relating to certain rare diseases, data relating to relatively small demographic groups.

Generative Artificial Intelligence (AI) models, which can generate new text on the basis of inputs, has the potential to offer a solution to both problems. The generative AI can solve the problem of privacy by producing synthetic EHR data which is uncorrelated to any individual patient and so cannot be re-identified and is thus able to be shared freely. Additionally, if the model is capable of generating labeled data, and particularly labeled data of specifically requested types, the datasets can be used to augment the underlying EHR data to allow for much stronger models than those trained on just the conventional and either unbalanced or unlabeled EHR data.

However, conventional approaches to generating synthetic EHR data using generative AI have typically not created labeled data, and more crucially have only generated data that is low-dimensional, consisting of aggregated medical codes into higher level categories or removing rare codes. Real EHR data, on the other hand, is extremely high dimensional and sparse.

Of all the EHR generation methods, rule-based approaches have proven to be the most effective in delivering practical value. These simple approaches either offer de-identification of real records by combining data across multiple patients in a sufficiently privacy-preserving way, simulation of patients within a complex yet constrained rule-based system, Bayesian probabilistic modeling of aggregated, non-temporal patient records, and/or other non-disclosed proprietary methods. Many of these systems can only produce synthetic patient data with limited capacity in realism and utility.

use Generative Adversarial Network (GAN)-based Methods use GANs, which involve a generator that creates realistic data, and a discriminator that decides if the data is real or fake. The GAN has been applied to patient record generation. However, GANs have limitations when generating sequential data like EHRs. In particular, GANs typically produce a single output (e.g., with no time connections). Accordingly, most GAN-based EHR generation methods aggregate EHR data into one time step and/or create a representation of EHR data.

GANs also struggle with high dimensional and sparse data like real-world EHR data, limiting all existing synthetic EHR GAN approaches to produce relatively low dimensional data through the aggregation of visits and medical codes or removal of rare codes. For example, there are a few methods in this category which generate longitudinal data.

While GANs may have the potential to be conditioned on external factors and labels, such as demographics or disease phenotype labels, the ability to do so has not been extensively explored in existing works on EHR generation. Moreover, there are only a limited number of approaches that can generate synthetic EHR data tailored to specific diseases.

Language generation models predict a next word based on the preceding words, thereby learning a probability distribution of languages. Similarly, EHR models predict the next visit based on past visits. This approach is particularly beneficial in accurately capturing the complex and high-dimensional nature of EHR data.

The Transformer architecture has revolutionized natural language processing and enabled the development of large, attention-based models like BERT and GPT. In particular, GPT relies on a stack of Transformer decoder blocks that use masking to predict the next set of probabilities in parallel, allowing for fast training and scalability. However, applying language models directly to EHR data poses unique challenges. Unlike natural language sequences, EHR data exhibits a hierarchical structure that must be captured, with medical codes associated with specific patient visits, and visits associated with individual patients. Additionally, EHR data contains heterogeneous elements, including demographic variables, structured medical codes, and numeric lab measures, not all of which are discrete tokens. Accordingly, language models are ill-equipped to deal with the peculiarities of EHR data.

Accordingly, to provide the necessary amount of utility for real-world use, there is a need for a generative AI model that can produce suitable high-dimensional synthesized EHR data.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

In embodiments, an EHR synthesis platform is provided. The platform is an autoregressive and explicitly probabilistic model for generating (e.g., synthesizing) EHR data. The platform may create labeled and full dimensional synthetic EHR datasets, utilizing a novel multi-granularity approach in order to model binary sequences of over a million variables. A generative AI model may receive, as training data, large sets of EHR data, including, but not necessarily limited to, outpatient claims datasets, inpatient hospital stay datasets, and/or any other available large datasets comprising EHR data.

Synthetic electronic health records (EHRs) that are both realistic and privacy-preserving offer alternatives to real EHRs for machine learning (ML) and statistical analysis. However, generating high-fidelity EHR data, particularly in its original, high-dimensional form, poses challenges. A Hierarchical Autoregressive Language mOdel (HALO) platform generates longitudinal, high-dimensional EHR, which preserves the statistical properties of real EHRs and can train accurate ML models without privacy concerns. The HALO platform generates a probability density function over medical codes, clinical visits, and patient records, allowing for generating realistic EHR data without requiring variable selection or aggregation. Extensive experiments demonstrated that the HALO platform can generate high-fidelity data with high-dimensional disease code probabilities closely mirroring (e.g., above 0.9 R² correlation) real EHR data. The HALO platform enhances the accuracy of predictive modeling and enables downstream ML models to attain similar accuracy as models trained on genuine data.

In one aspect, the present disclosure relates to a platform (e.g., a method, system, and/or computer program product) for creating synthetic electronic health records, the platform being configured to perform operations including receiving EHR data and encoding the received EHR data as a plurality of fixed-length vectors. The platform provides the fixed-length vectors to a machine learning model, and produces a plurality of visit history representations. The platform applies code information corresponding to medical codes associated with each visit history to form appended visit histories. One or more appended visit histories are provided to one or more masked linear layers to produce a probability matrix comprising probabilities for each code for each visit. The platform produces one or more synthetic EHRs based on the probability matrix.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 illustrates a block diagram of an architecture of a platform for synthesizing high-dimensional longitudinal Electronic Health Records (EHRs) using a deep learning language model consistent with the present disclosure;

FIG. 2A is a diagram showing the formatting of a single patient visit in an EHR;

FIG. 2B is a diagram showing the formatting of EHR data for input to the platform of FIG. 1;

FIG. 3A shows code probability plots comparing the performance of the platform of FIG. 1 to that of various baseline models;

FIG. 3B shows code probability plots comparing the performance of the platform of FIG. 1 to that of various baseline models;

FIG. 3C shows code probability plots comparing the performance of the platform of FIG. 1 to that of various baseline models;

FIG. 4A shows a graph comparing outpatient EHR visit gap probability densities of synthetic EHR data produced by the platform of FIG. 1 and real EHR data;

FIG. 4B shows a graph comparing average visit gaps of synthetic EHR data produced by the platform of FIG. 1 and real EHR data;

FIG. 4C shows a graph comparing lab presence probabilities of synthetic EHR data produced by the platform of FIG. 1 and real EHR data;

FIG. 4D shows a graph comparing average lab values of synthetic EHR data produced by the platform of FIG. 1 and real EHR data;

FIG. 5 is a flow chart of a method for synthesizing high-dimensional longitudinal EHRs using a deep learning language model; and

FIG. 6 is a block diagram of a system including a computing device for performing the method of FIG. 5.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely to provide a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such a term to mean based on the contextual use of the term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subject matter disclosed under the header.

The present disclosure includes many aspects and features. Moreover, while many aspects and features relate to, and are described in, the context of EHR dataset synthesis, embodiments of the present disclosure are not limited to use only in this context.

### I. PLATFORM OVERVIEW

This overview is provided to introduce a selection of concepts in a simplified form that are further described below. This overview is not intended to identify key features or essential features of the claimed subject matter. Nor is this overview intended to be used to limit the claimed subject matter's scope.

Electronic Health Records (EHRs) are conventionally viewed as multi-granular longitudinal records. For each patient, the patient is represented by a plurality of visits. In turn, each visit is represented by a series of medical codes characterizing one or more diagnoses given, procedures performed, and/or medications prescribed during that visit. Additional information regarding patient demographic information, long term condition or disease labels, lab tests performed, and time between visits may also be included in the EHR.

A Generative Adversarial Network. (GAN) may utilize adversarial training between a generator model which seeks to produce realistic looking data and a discriminator model which seeks to correctly determine whether passed data is real or fake. GANs have risen in popularity in other arts such that it makes sense that they have been a popular choice for the EHR generation domain as well. However, GANs are an imperfect solution to the problem of generating sequential data, such as EHRs. In particular, GANs typically produce a single output and so have resulted in much of the work within the field modeling either EHRs which are aggregated into a single time step and/or latent EHR representations. Moreover, GANs struggle with high dimensional, sparse data, causing existing synthetic EHR approaches to produce relatively low-dimensional data through the aggregation of visits, combination of codes, removal of rare codes, and/or other dimensionality reductions. Additionally, while GANs can be conditioned on external factors and labels, GANs have not been used for demographics data or chronic condition/disease labels in EHRs.

The approaches which have been most successful in providing utility to this point are those which are fully data or rule-based. These approaches either offer de-identification of real records by combining data across multiple patients in a sufficiently privatizing way, simulation of patients within a complex yet constrained rule-based system, Bayesian probabilistic modeling of aggregated, or non-temporal patient records. Regardless, these systems are constrained by definition and so have a limit on both their realism and utility.

Thus, the most promising route forward is via machine learning models which are able to successfully model the longitudinal nature of EHRs. Conventional uses of machine learning have achieved strong results in data augmentation of longitudinal records, but are conditioned completely on existing patient health records and only fill in some masked visits, such that they do not offer any privacy guarantees. Moreover, such models merely augment existing data rather than offering full modeling and generative capabilities. Some models may produce longitudinal records and include continuous variables, but are limited to time series lab information across a single ICU visit rather than diagnosis, prescription, and procedure history across multiple visits as EHRs are typically defined. Some models have succeeded in achieving this sequential generation by conditioning on representations of past patient visits to produce current visits using a GAN and/or a variational autoencoder (VAE). However, these models still perform certain preprocessing steps that limit the scope of the vocabulary in terms of aggregating medical codes and/or remove rare code occurrences.

Language models are similar in some ways to generative models for EHRs. They seek to learn an explicit probability distribution of languages or sequences of words by predicting the probabilities of the next token or word based on the previously seen tokens in much the same way as longitudinal models of EHRs seek to predict the next visit based on the visits that have been seen to that point. This explicit probability output is additionally valuable in both modeling and evaluating the underlying data distribution directly as opposed to the indirect approach of GAN-based models. Since the popularization of the Transformer architecture, most popular and state of the art language models have been large, attention-based models, such as BERT and GPT. In particular, the GPT architecture utilizes large stacks of Transformer decoder blocks that mask the inputs in order to allow for the prediction of the next set of probabilities at each time step for an entire sequence in parallel, yielding fast training and thus permitting the model to be scaled up as large as possible.

In embodiments, a platform for generating high-dimensional EHR data in its native form may be a Hierarchical Autoregressive Language Model (HALO). This model takes an autoregressive and probabilistic approach. In embodiments, this model may capture the hierarchical distribution of EHR records and their temporal relationships. Using a hierarchical approach to model binary sequences of over a million variables, HALO can efficiently learn and represent complex patterns in EHR data.

The HALO model works by utilizing a pair of modules to represent both the visit- and code-level structures of a patient record. First, the model uses a coarse, visit-level module to factorize the probability along each of a patient's visits and to efficiently process and represent a patient's past medical history. A fine, code-level module may generate each variable in a given visit based on the past history and/or the previous variables in the same visits for maximum intra-visit cohesion.

Embodiments of the present disclosure may comprise methods, systems, and a computer readable medium comprising, but not limited to, at least one of the following:
A. A Visit-Level Module
B. A Code-Level Module

Details with regards to each module are provided below. Although modules are disclosed with specific functionality, it should be understood that functionality may be shared between modules, with some functions split between modules, while other functions duplicated by the modules. Furthermore, the name of each module should not be construed as limiting upon the functionality of the module. Moreover, each component disclosed within each module can be considered independently, without the context of the other components within the same module or different modules. Each component may contain functionality defined in other portions of this specification. Each component disclosed for one module may be mixed with the functionality of other modules. In the present disclosure, each component can be claimed on its own and/or interchangeably with other components of other modules.

The following depicts an example of a method of a plurality of methods that may be performed by at least one of the aforementioned modules, or components thereof. Various hardware components may be used at the various stages of the operations disclosed with reference to each module. For example, although methods may be described to be performed by a single computing device, it should be understood that, in some embodiments, different operations may be performed by different networked elements in operative communication with the computing device. For example, at least one computing device 600 may be employed in the performance of some or all of the stages disclosed with regard to the methods. Similarly, an apparatus may be employed in the performance of some or all of the stages of the methods. As such, the apparatus may comprise at least those architectural components as found in computing device 600.

Furthermore, although the stages of the following example method are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in orders that differ from the ones disclosed below. Moreover, various stages may be added or removed without altering or departing from the fundamental scope of the depicted methods and systems disclosed herein.

Consistent with embodiments of the present disclosure, a method may be performed by at least one of the modules disclosed herein. The method may be embodied as, for example, but not limited to, computer instructions which, when executed, perform the method. The method may comprise the following stages:
Receiving electronic health record (EHR) data;
Encoding the EHR data as a fixed-length vector;
Providing the vector to a machine learning model to produce visit history representations that include collective information of all prior visits;
Appending code information to one or more of the visit history representations to for appended visit history representations further including code information for the current visit;
Providing the appended visit history representations to one or more masked linear layers to produce a probability matrix comprising probabilities of each code for each visit after the start visit built from the appended visit histories; and
Generating a synthetic EHR based on the probability matrix, wherein the synthetic EHRs may be used to train one or more further machine learning models.

Although the aforementioned method has been described to be performed by the platform 100, it should be understood that computing device 600 may be used to perform the various stages of the method. Furthermore, in some embodiments, different operations may be performed by different networked elements in operative communication with computing device 600. For example, a plurality of computing devices may be employed in the performance of some or all of the stages in the aforementioned method. Moreover, a plurality of computing devices may be configured much like a single computing device 600. Similarly, an apparatus may be employed in the performance of some or all stages in the method. The apparatus may also be configured much like computing device 600.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

### II. PLATFORM CONFIGURATION

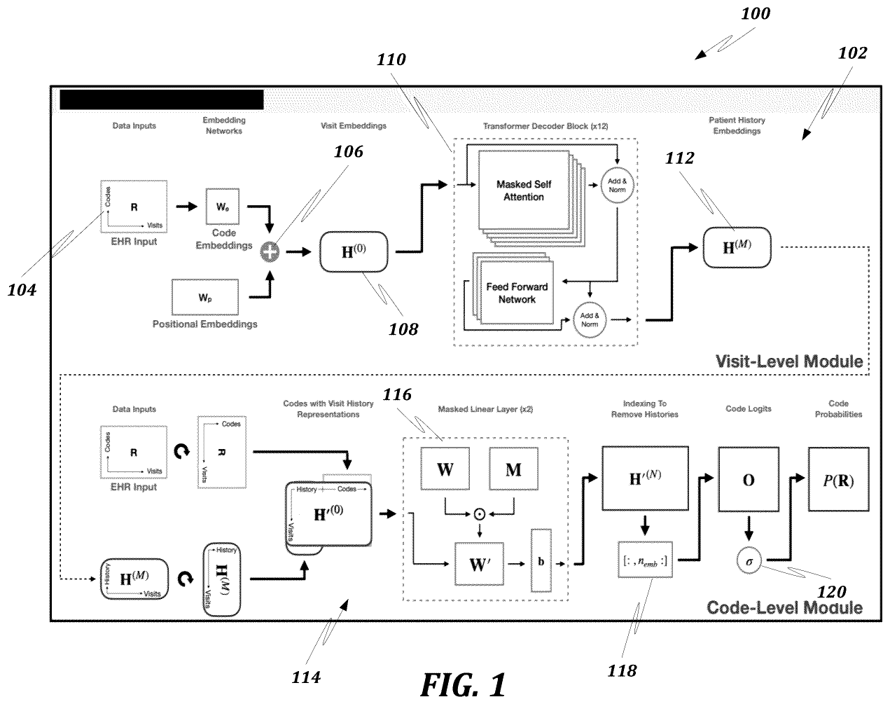

FIG. 1 illustrates one possible operating environment through which a platform consistent with embodiments of the present disclosure may be provided. By way of non-limiting example, a record data generation platform 100 may be hosted on, for example, a cloud computing service. In some embodiments, the platform 100 may be hosted on a computing device 600. A user may access platform 100 through a software application and/or hardware device. The software application may be embodied as, for example, but not be limited to, a website, a web application, a desktop application, and a mobile application compatible with the computing device 600.

Structured EHRs are multi-level longitudinal records, where each patient is represented by a sequence of visits. Each visit may be characterized by a set of medical codes, reflecting the diagnoses, procedures, and medications administered during that visit. Additional patient information, such as demographics, disease phenotype labels, lab test results, and inter-visit time, may also be included.

EHR Data for a patient may include a patient record R as a sequence of visits over time such that:

R=V⁽¹⁾,V⁽²⁾, . . . ,V^{(T)}

where each visit V(t) contains a varying number of medical codes m₁^{(t)}, m₂^{(t)}, . . . ,

$${m}_{\vert {V}_{C}^{( t )} \vert}^{( t )} ∈ C ,$$

lab values l₁^{(t)}, l₂^{(t)}, . . . ,

$${l}_{\vert {V}_{L}^{( t )} \vert}^{( t )} ∈ L ,$$

and the inter-visit time gap g^{(t)}. Accordingly, C represents the set of all medical codes in our vocabulary, including diagnoses, procedures, and medications; and L represents the set of all labs. In some embodiments, a patient record may include some static information S containing demographic data such as (but not limited to) gender, race, and birth year and/or a set of disease phenotype labels D indicating one or more major and persistent disease conditions.

To allow input of the patient record to the record data generation platform, R, S, and D are converted into a matrix representation R. Specifically, we build R=[vₛ, vₗ, v₁, . . . , v_{T}vₑ], a matrix containing a sequence of the vector representations for each of the patient's T visits, a preceding start visit, a label visit and a succeeding end visit.

The start visit vₛ is a one-hot vector containing a special start code added to C to signify the start of the record. The label visit vₗ contains one or more special codes added to C, representing demographic and chronic disease phenotypes from S and D, respectively. As a non-limiting example, the label visit may include codes representing (as non-limiting examples) the gender, racial and ethnic groups, birth year of the patient, any chronic condition and/or outcome labels associated with the patient, any genomic information and/or anything else on which EHR synthesis may be conditioned.

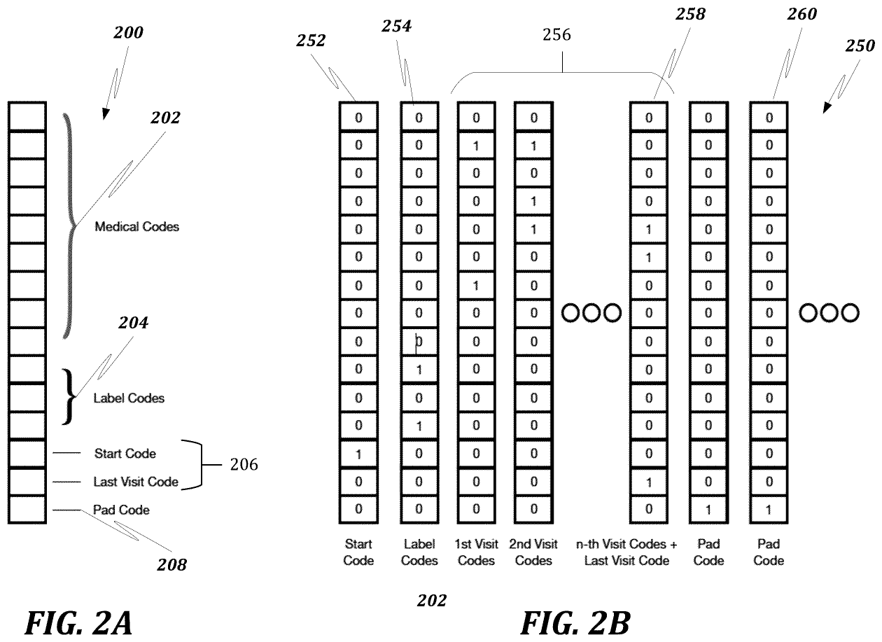

As shown in FIG. 2A, each visit may be represented as a multi-hot vector 200. The vector 200 may be made up of one or more of: indices for medical codes 202, static label codes 204 (e.g., to identify demographics and disease phenotypes), and/or special codes 206 (e.g., describing the shape and temporal ordering of the patient's visit), including (but not limited to) a start code, an end code, a last visit code, and/or the like. In some embodiments, the vector 200 may include one or more pad codes 208 to adjust the matrix length such that each matrix has the same length (e.g., the same number of visit vectors). Thus, each visit vₜ∈R^{|C|} may be represented as a multi-hot binary vector including medical codes and/or lab values present in that visit, and/or an inter-visit gap representing a time between that visit and an immediately prior visit. To represent continuous lab values and visit gaps in a discrete form, a granular discretization process may be used. For example, multiple range codes may be added to C for each lab test and for the intervals between visits. By converting all medical information into binary variables, Cₜⁱ represents the presence of the i^{th} code in C in the t^{th} visit of the patient record R.

The end visit vector vₑ is a one-hot vector containing a special end code added to C to signify the end of the record. In some embodiments, the end visit vector serves a similar purpose to a stop token in natural language generation. In some cases, a stop code may be included in a final patient visit in place of or in addition to adding the end visit vector. This may enable a generative model to learn when to terminate a record and/or allow for R to be padded by adding columns to a constant length for batch input without altering vector content.

FIG. 2B shows the format of the EHR representation 250. In particular, the EHR 250 is represented as a matrix constructed as a series of temporally ordered vectors, including a start vector 252, a label vector 254, a plurality of visit vectors 256, one or more optional pad vectors 258, and an end vector 260.

In embodiments, the platform 100 may model the probability of patient record R, P(R), via a hierarchical autoregressive model, which utilizes both visit- and code-level structures of a patient record. First, the platform may factorizes the probability along the visit level using the autoregressive identity:

$$P ( R ) = P ( {v}_{s} , {v}_{l} , {v}_{1} , ... , {v}_{T} , {v}_{e} ) = P ( {v}_{s} ) P ( {v}_{l} \vert {v}_{s} ) P ( {v}_{1} \vert {v}_{s} , {v}_{l} ) ... P ( {v}_{e} \vert {v}_{s} , {v}_{l} , ... , {v}_{T} )$$

to produce a coarse autoregressive sequence. The platform 100 may continue to factorize the probability of visits further along the code level by converting:

P(vₜ|vₛ, . . . ,vₜ₋₁)=P(cₜ¹|vₛ, . . . ,vₜ₋₁)P(cₜ²|vₛ, . . . ,vₜ₋₁) . . . P(cₜ^{C}|vₛ, . . . ,vₜ₋₁)

into what a fine autoregressive sequence. This final probability is then rewritten as the product:

$$P ( R ) = {\prod }_{t} {{\prod }^{C}}_{i} P ( {c}_{t}^{i} \vert {v}_{s} , ... , {v}_{t - 1} , {c}_{t}^{1} , ... {c}_{t}^{i - 1} )$$

where the probability of each code is based on each of the previous visits and each of the previous codes in the current visit. This multigranularity approach may help to enable modeling of high-dimensional sequences of many binary variables per record. This is achieved, at least in part, by grouping prior information into significantly fewer multivariate time steps for previous visits, while retaining the full autoregressive modeling capability for each current visit. The HALO platform 100 is designed to reflect this powerful yet compact model, with a powerful and efficient structure divided into two distinct granularity levels: visit level and code level. This allows for each code to be conditioned on all previous visits and the past codes of the current visit.

Embodiments of the present disclosure provide a software and hardware platform comprised of a distributed set of computing elements, including, but not limited to:

A. Visit-Level Module

The platform 100 may include a visit-level module 102. The visit-level module 102 may include hardware and/or software for generating visit data for a synthesized health record. In embodiments, the generated visit data may be statistically similar to visit data included in at least one EHR used as input. Accordingly, the visit level module may receive, as an input, actual EHR data associated with a patient. The output of the visit level module 102 may include a synthetic listing of patient visits that is statistically similar to one or more input EHRs, but it not identifiable to any single patient.

The visit level module 102 may receive an input comprising EHR data. The EHR data may be received via an interface 104. In some embodiments, the EHR data may be represented as a matrix or series of vectors (e.g., the EHR matrix 250). Alternatively, the visit level module may transform the EHR data into a vector representation thereof.

The received EHR data may be processed by one or more first embeddings 106. An embedding is hardware and/or software that maps a discrete variable to a vector of continuous numbers. The embeddings 106 may reduce the dimensionality of categorical variables and meaningfully represent categories in the transformed space. For example, the visit level module 102 may include code embeddings and positional embeddings as shown in FIG. 1.

After being processed by the first embeddings 106, the data may be aggregated and communicated to a set of visit embedding 108 to from visit data. The visit data produced by the visit embedding 108 is real data associated with a patient. The visit data may be used as input for a machine learning model 110. The machine learning model 110 may be, for example, a transformer model, as shown in FIG. 1. The Transformer architecture has an encoder-decoder structure, but does not necessarily rely on recurrence and convolutions in order to generate an output. The encoder of the Transformer architecture may map an input sequence to a sequence of continuous representations, which is then fed into a decoder. The decoder may receive the output of the encoder together with the decoder output at the previous time step to generate an output sequence. At each step the model is auto-regressive, consuming the previously generated symbols as additional input when generating the next.

The output of the machine learning model 110 may then be further processed using an output embedding 112. For example, the output embedding 112 may include a patient history embedding used to form the output of the machine learning model 110 into a synthesized patient visit history. The synthesized patient history may be output from the visit level module 102.

To form the coarse, visit-level granularity, a stack of M transformer decoder blocks, similar to those shown to be effective in the high-dimensional domain of natural language processing, may be used as the machine learning model 110. The transformer decoder blocks may be used to generate a sequence of visit-level histories, where the t^{th} element in the sequence, hₜ^{(M)}∈ [symbol] , is an embedding that represents all of a patient's medical history through their t^{th} visit. Those histories then combine to form H^{(M)}∈ [symbol] (where the 3 in T+3 includes the start, label, and end visits).

To encode each of the multi-hot visit representations [v₁ . . . vₙ] into a fixed-length vector in [symbol] , an embedding layer is used. The embedding layer may include two trainable parameter matrices: a code embedding matrix W_{c} and a positional embedding matrix Wₚ. The code embedding matrix maps each visit code to a dense vector representation; the positional embedding matrix captures the relative position of each visit in the sequence. A decoder model consisting of M (a particular number, e.g., 12) transformer decoder blocks to generate a series of visit history representations, which summarize the information contained in all previous visits in the coarse, visit-level sequence. One or more (e.g., each) of the transformer decoder blocks may employ masked multihead self-attention, which allows the model to attend to all previous visits while preventing information leakage from future visits. This process is written more formally as:

H⁽⁰⁾=RWₑ+W_{P}

H^{(m)}=transformer_block(H^{(m-1)})∀m∈[1,M]

where R∈ [symbol] is the patient record matrix representation, Wₑ∈ [symbol] is the code embedding matrix, Wₚ∈ [symbol] is the positional embedding matrix (to recapture the position and order of the sequence of visits), and each transformer block is based on a decoder block from the original transformer architecture.

Thus, having processed the multi-hot patient visits through the initial, coarse visit-level module of our architecture, a sequence of visit history representations H^{(M)} is produced. The visit history representations represent the collective information of all previous visits up to each time step. The visit history representations may provide a compressed summary of the patient's visit history, enabling downstream modules to make predictions based on the patient's medical trajectory.

B. Code Level Module

The platform 100 may include a code-level module 114. The code-level module 114 may include hardware and/or software for generating code data to be associated with the synthesized visit data output for a synthesized health record. The output of the visit level module 102 may be provided to the Code level module 114 as an input, along with the EHR data. In some embodiments, the synthesized visit matrix output by the visit level module 102 and/or the input EHR data matrix may be transposed such that both matrices store data so that each visit is represented by a discrete row. Thereafter the matrices may be concatenated such that, for each row (e.g., each visit) the history data from the synthesized visit matrix is followed by the code data from the EHR data matrix. In this way, each visit is associated with both history and diagnostic codes.

The concatenated matrix may be provided as input to a set of one or more of masked linear layers 116, and then indexed to remove histories at 118, producing diagnostic code logits (e.g., un-normalized output from the one or more masked linear layers 116). Thereafter, the code logits can be input to a normalizer 120 to produce normalized code probabilities.

Thus, to construct the input for the fine, code-level module 114, the visit history embedding outputs from the visit level module 102 may be offset and concatenated with the original record input, R. Specifically, the first T+2 visit histories may be appended to the last T+2 visit representations [vₗ, v₁, . . . , v_{T}, vₑ] to create H′⁽⁰⁾. Each of the T+2 inputs in H′⁽⁰⁾ has a representation of the history of all the previous visits and the codes of the current visit, mirroring both the visit and code priors. The final input representation H′⁽⁰⁾ has a size [symbol] .

To model the distribution of each [symbol] H⁽⁰⁾ may be fed through a number N (e.g., two) of masked linear layers which maintain the same dimensionality and use upper triangular masking of the weight matrix to preserve the autoregressive property of the probabilities (and have a ReLU activation function between layers). The masked linear layers are able to efficiently model the high-dimensional, intra-visit patterns where other sequential approaches (e.g., additional recurrent or transformer modules) would run out of memory. The probabilities are generated formally by:

H′⁽⁰⁾=offset_and_concat(H^{(M)},R)

H′⁽⁰⁾=masked_linear(H′⁽ⁿ⁻¹⁾)∀n∈[1,N]

O=sigmoid(H′^{(N)}[:,n_{emb}:])

where the submatrix indexing at the end removes the visit-level history embedding portions of each vector to extract just the code probabilities, and the masked linear layers are achieved by:

H′⁽ⁿ⁾=max(0,H′⁽ⁿ⁻¹⁾)(W⁽ⁿ⁾⊙M)+b⁽ⁿ⁾)

where the max function may be omitted for at least the final fine layer (and wherein the sigmoid function may be used in place of the max function), ⊙ is an element-wise matrix multiplication, M∈ [symbol] is the upper triangular masking matrix (e.g., a matrix having ones in the upper triangular portion and zeros in the lower portion) to preserve the autoregressive property, and W⁽ⁿ⁾ ∈ [symbol] are trainable parameters of the module.

The output O∈ [symbol] represents a matrix of probabilities of each code for each visit after the start visit. In embodiments, the matrix is built from the visit histories and each previous code in the same visit.

The HALO platform 100 may be trained using, as a non-limiting example, the binary cross-entropy loss function over each medical code (treating the problem as a multi-label classification problem). In some embodiments, masking may be applied such that the start visit as well as any padded visits (of all zeros) do not contribute to the entropy loss.

In this way, the platform 100 can utilize an autoregressive multi-granularity approach which analyzes at both the visit and code level to generate next code probabilities based on the history of all previous visits as generated through the transformer decoder layers and the previous codes in the current visit through the masked linear layers.

### Generation Process

The trained HALO model enables multiple strategies to convert the outputted probability matrix to real patient records. The architecture of the HALO model operates to generate a probability distribution over the entire record R, but the actual sampling process may be performed sequentially, one code at a time. During generation, the input record R′, a subset of the to-be-completed record filled up through the i-th code in the t-th visit, may be fed into the model, generating the probability P(cₜ^{i+t}). This probability is sampled to produce cₜ^{i+t}, which is added to R′$ before repeating the process for the next code. This is the basic process of generating a new, synthetic EHR record. This sampling process may be optimized in a number of ways.

An EHR consists of an extremely long binary sequence. One of the benefits provided by HALO is in grouping past visit representations through the visit-level module to allow the processing of the long binary sequence with full autoregressive dependencies while maintaining the nuances of intravisit, code-level modeling. This efficient representation does not avoid the long, code-by-code generation process. However, the records are also very sparse. For example, the records may include on the order of tens of medical codes selected out of thousands or tens of thousands at each visit. Generation may be sped up by generating probabilities for all of the rest of the codes in the current visit, assuming that they are all zero and sampling from all of the probabilities simultaneously. The next positively sampled code may be found and skip directly to that location in the generation process by noting that all previous codes will remain zero and so its probability is still correct. In this way, we are able to group code generations as well to greatly speed up generation.

Random or pseudo-random sampling throughout the generation process may be used to add diversity, as sampling the same sequence from the same starting point will yield different results. However, it is still a tunable process. The trained model is assumed to have learned an approximation of the true record distribution and so purely random sampling is the most accurate recreation of real patients. Nonetheless, the temperature can be used to augment the probabilities to increase the probability of common and/or rare codes and patients. Thresholding helps to remove variability and may convert the simulated codes to predictions. Finally, desired types of patients can also be simulated by mandating certain codes, especially early in a patient's history.

### Conditional Generation

In some embodiments, the platform 100 generates EHR data by using demographics S and chronic disease phenotypes D as labels, which are represented in our label vocabulary and applied to individual visits, as shown in FIG. 2. These labels are selected based on their relevance to downstream use cases. Each label may be represented as a binary variable in vₗ, indicating the presence of the corresponding disease or demographics group indicator. These indicators are defined by concepts such as (but not limited to) specific categories of genders, races, ethnicity, age groups, and the like. Other labels of interest including, as non-limiting examples, various biomarkers, patient outcomes, or even abstract patient embeddings may also be included in the labels.

### Unconditional Generation

In some embodiments, the platform 100 generates EHR data with conditional labels by incorporating a “label visit” in the data format, as illustrated in FIG. 2. This format enables easy generation of labeled and conditional data, which are highly valuable for using synthetic data in machine learning tasks and as an augmentation tool, particularly for rare cohorts. However, this formatting (e.g., the presence of the label visit) is optional. In some embodiments, the “label visit” can be removed from the EHR representation, and the platform 100 can be trained to generate unconditioned EHRs without any modification.

### Generation of Continuous Variables

The platform 100 can generate not only medical codes but also continuous variables, such as lab values and temporal gaps between visits. The availability of these additional variables in the generated data depends on their presence in the original dataset used for training. For example, an outpatient EHR dataset may include time between visits, while an inpatient EHR dataset includes lab values.

In conventional models, continuous values were typically generated using either GANs, which lack the autoregressive probabilistic modeling of the HALO platform 100, or value predictors (such as time series analysis models), which often produce average values with insufficient variance. To overcome these limitations, the platform 100 may model continuous variables within the healthcare domain by discretizing lab values and temporal gaps into clinically equivalent buckets. The resulting binary variables are included in the model's context, denoted as C, before being converted back to continuous values through random uniform sampling within the corresponding bucket range. This approach allows the HALO platform 100 to generate more realistic and diverse continuous variables than previous methods.

More specifically, to generate discrete versions of continuous variables, such as lab values and temporal gaps, the range of each variable is divided into a plurality of “buckets”, as represented by the values b₁, b₂, . . . ,

$${b}_{\vert {l}_{j}^{( t )} \vert}$$

where

$$\vert {l}_{j}^{( t )} \vert$$

refers to the number of buckets required. The bucket ranges may be determined by seeking advice from clinicians on practical ranges, creating granular but equivalent groupings, and/or using a histogram construction algorithm. The same approach may be applied to temporal gaps as well.

For example, a heart rate lab test may have possible values ranging from 0-400 beats per minute could be broken down into twenty different buckets splitting the overall span into smaller ranges which offer substantially the same medical meaning for all their contained values. This breakdown could include bucket b₁, corresponding to a heart rate (0, 40) and bucket b₇ corresponding to a heart rate (90, 100). These buckets are used to convert the single continuous variable into many binary variables. Whenever the continuous variable is present in the original EHR, a single one of the variables representing a corresponding bucket is set to 1 with the rest remaining 0. For instance, if a patient has a heart rate lab measurement of 93 bpm during a particular visit, the seventh of the new heart rate variables (the variable corresponding to bucket b₇) for that particular visit would be set to 1 and the other variables (corresponding to the other buckets for that test) would be set to 0. If there was no such lab measurement in the visit, the variables corresponding to all buckets for that test would be set to 0.

The binary variables representing the test are added into the wider code vocabulary C and treated in the same way as all of the other medical codes in the vocabulary by the HALO platform 100 during learning and generation. After generation, the specific lab values and inter-visit gaps may be converted back into a continuous value by uniformly sampling from the corresponding bucket range.

This discretization allows the HALO platform 100 to maintain the same powerful and probabilistic modeling process, matching the probabilistic variance of real continuous values in the same way the variance of medical code presences is matched. By building appropriately granular buckets (e.g., buckets where the values within the bucket offer substantially the same medical significance), the platform 100 can avoid losing meaningful information and maintain a full representation of a patient.

### III. PLATFORM EVALUATION

In embodiments, the generation task for the platform 100 is to create R′, a synthetic patient record that is statistically similar to and offers the utility of R without any one-to-one mapping to a real patient. As described above, the platform 100 does this, at least in part, by learning distribution P(R).

The platform 100 may be evaluated and compared to several conventional methods, comprising both recently proposed models and other logical autoregressive model architectures, on a series of experiments on both outpatient and inpatient EHR datasets. To maintain the fidelity of the original EHR data, the experiments focus on synthesizing original granular medical codes without aggregating or combining codes.

Two datasets may be used for platform evaluation: An outpatient EHR dataset is from a large real-world US claims data store. The outpatient dataset contains 929,268 patients and binary labels for 11 chronic diseases. This yields a final real-world outpatient EHR dataset with an average of 34.16 visits per record and 3.52 codes per visit with 9,882 unique ICD-10 codes. An inpatient EHR dataset is from the MIMIC-Ill ICU stay dataset. The inpatient dataset contains 46,520 patients with 25 disease phenotype labels as defined by the MIMIC benchmark. This dataset has an average of 1.26 visits per record and 15.11 codes per visit with 6,841 unique ICD-9 codes. Note that this includes patients with just a single visit (the Code-Level Module of the platform 100 allows it to be very effective on single-visit patients).

The conventional platforms against which the platform 100 is measured (and necessary alterations to those platforms) include:
HALO-Coarse: This baseline is an ablation baseline consisting of just the coarse, visit-level granularity module of the full HALO architecture (e.g., the platform 100). It generates each code probability based on all previous visits (grouped into a multi-hot representation) but with-out the fine, inter-visit modeling such that P(cᵢⁱ) is modeled by P(cᵢⁱ|v₁ . . . , vₜ₋₁) instead of P(cᵢⁱ|v₁ . . . , vₜ₋₁, cᵢ¹, . . . , cᵢⁱ⁻¹). It includes a plurality (e.g., 12) transformer decoder blocks augmented to support multi-hot in place of or in addition to one-hot inputs and outputs within an embedding layer and final activation layer.
GPT Model: the GPT model applied to the EHR data without any augmentation to support multi-hot inputs and outputs, but instead with the conversion of EHRs to a fully one-hot sequential representation. The GPT model was reduced to 3 blocks from 12 to fit into memory.
LSTM EHR Model: A deep, autoregressive Long Short Term Memory (LSTM) model, adapted to generate structured patient records rather than unstructured text as it had previously been utilized, which uses LSTM blocks instead of transformer decoder blocks.
SynTEG: A Generative Adversarial Network (GAN)-based model that uses a transformer and LSTM-based encoder model to generate embeddings of EHRs up to a given visit before feeding those embeddings into a conditional GAN which generates the next visit.
EVA: a Variational AutoEncoder (VAE)-based model which uses a bidirectional-LSTM encoder and Convolutional Neural Network (CNN)-based decoder (using deconvolutions to expand the latent encoding to the proper temporal dimension and then masked, diluted 1D convolutions to build the records in an autoregressive manner). The EVA output was converted from one-hot code combinations to multi-hot code probabilities to allow for greater representative power.

In a first analysis, the platform 100 and baseline platforms predicted probabilities and outputs of the test set are evaluated. In this phase, the performance of the HALO platform 100 is compared to that of two multi-hot language model baselines, namely HALO-Coarse and LSTM. These baselines explicitly generate a probability distribution without accessing the entire input. The other baseline models (e.g., SynTEG, EVA, and GPT) cannot be directly compared in this task because these models do not make a single probability prediction for each code within the visit.

The first evaluation assesses the capability of the models to predict the presence of potential medical codes, given a patient's past medical history and the previous codes from the current visit. This evaluation demonstrates the ability for a model to learn patterns from the patient population, and/or the potential for the model to perform well in various patient simulation and extension applications.

TABLE 1
Test Set Modeling Metrics

| | Outpatient EHR | | | Inpatient EHR | | |
|---|---|---|---|---|---|---|
| | BCE Loss | F1 Score | PP Per Code | BCE Loss | F1 Score | PP Per Code |
| LSTM | 7.744 × 10⁻⁴ | 0 | 660.204 | 2.600 × 10⁻⁴ | 0.193 | 74.565 |
| HALO-Coarse | 1.631 × 10⁻⁴ | 0.829 | 3.927 | 2.019 × 10⁻⁴ | 0.343 | 28.448 |
| HALO | 1.624 × 10⁻⁴ | 0.828 | 3.903 | 1.932 × 10⁻⁴ | 0.414 | 24.664 |

Table 1 shows the results of the first evaluation, showing that the HALO platform 100 outperforms the two compared language model architectures. The LSTM baseline model struggled with the complexity and size of the outpatient EHR dataset, while the HALO platform 100 performed comparably to the HALO-Coarse ablation baseline model. In the inpatient EHR setting, where the visits are shorter but contain more codes, the multi-granularity approach of the HALO platform 100 proved to be highly effective. Specifically, the model achieved a notable 4% reduction in binary cross-entropy (BCE) loss and a 17% increase in F1 Score on test data when compared to the single granularity HALO-Coarse model. Both HALO models significantly outperformed the LSTM baseline in this setting. These results highlight the significant value of the multi-granularity approach in handling the complex and diverse nature of medical codes in different EHR settings.

Additionally, Table 1 shows perplexity, which evaluates the probability or likelihood of the test set as quantified by a model trained on the training set, normalized by the number of medical codes in a patient's medical record (or equivalently number of medical codes in R). Perplexity is a metric found commonly in the wider generative modeling domain, especially on the task of natural language generation, though not previously used for synthetic EHR generation. Perplexity is defined mathematically by:

$$PP ( D ) = \sqrt[N]{\frac{1}{P ( D )}} = \sqrt[N]{\frac{1}{P ( {R}^{( 1 )} , ... , {R}^{( \vert D \vert )} )}} = \sqrt[N]{\frac{1}{P ( {R}^{( 1 )} ) ... P ( {R}^{( \vert D \vert )} )}}$$

where D is the test dataset and R(t) is the t^{th} record in D. Alternatively, the perplexity values can be calculated by summing their log probabilities, using the equivalent form:

$$PP ( D ) = exp ( - \frac{1}{N} {\sum }_{R ∈ D} log P ( R ) )$$

The normalized value then also corresponds to how many of the different normalizing units (e.g., medical codes) one would have to randomly pick between on average to achieve the same probability. The results of the perplexity evaluation are shown in Table 1 as well. We see similar results as with the classification evaluation with both HALO and HALO-Coarse performing very well on the outpatient EHR dataset (with HALO performing slightly better). The LSTM baseline model struggles in perplexity as well. The HALO platform 100 easily outpaces both baseline methods in this likelihood evaluation for the inpatient EHR dataset, producing a 13% lower perplexity per present code as compared to the HALO-Coarse model (e.g., without the inter-visit modeling). Thus, in both of these test set evaluations, we see that HALO is much more effective in terms of modeling the underlying distribution of EHRs.

In a second analysis. the statistical similarity of the synthetic data (e.g., the datasets generated by the platform 100 and the baseline models) and real data is evaluated. For each model, a synthetic dataset of the same size as the training dataset is generated. A comparison is made of unigram and/or bigram (both within the same visit and across consecutive visits) probabilities for each unique code and pair of codes within the real and synthetic datasets.

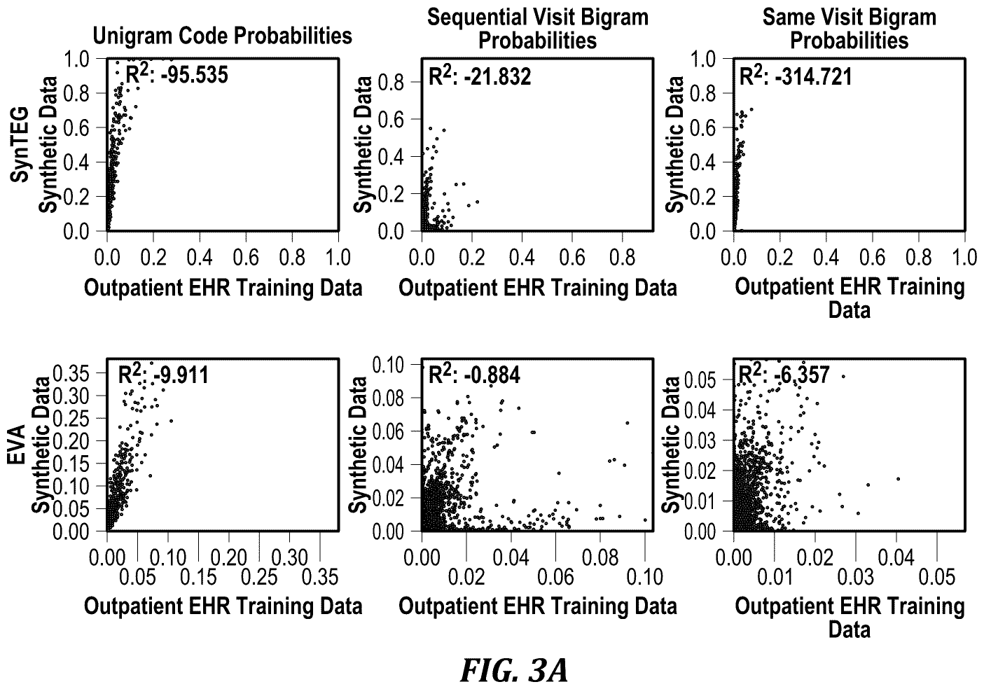

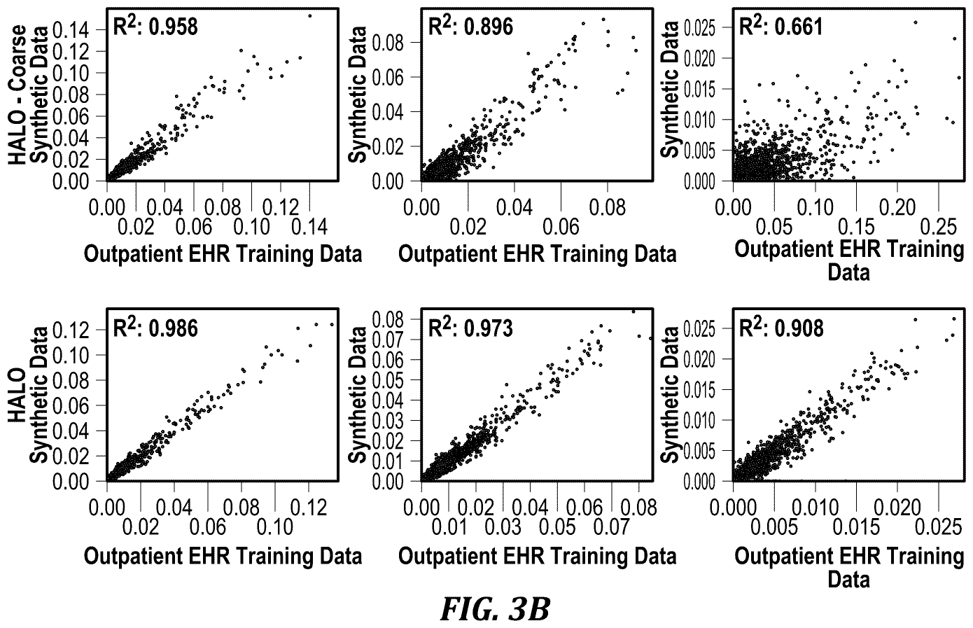

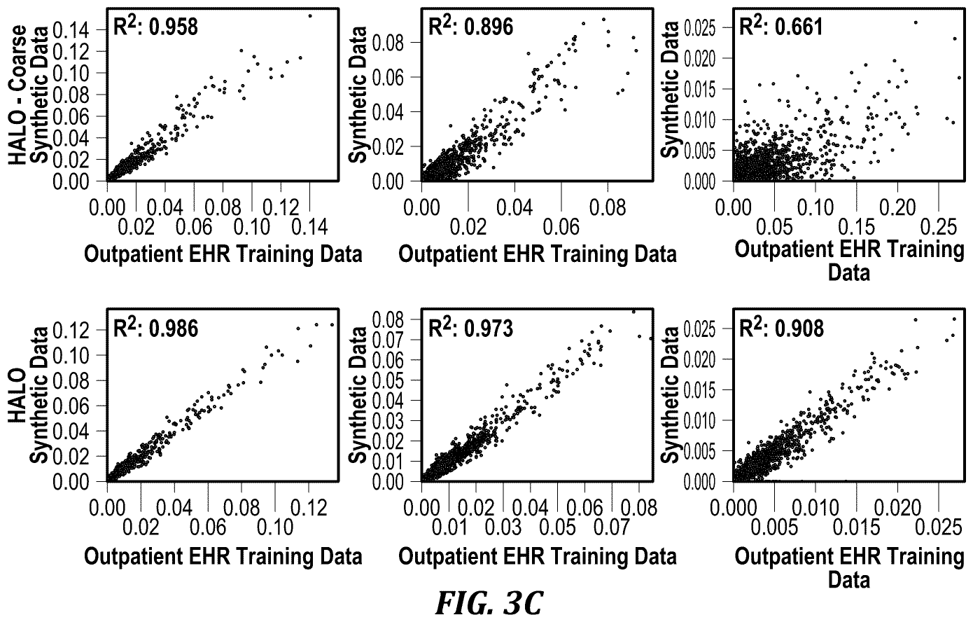

The data is evaluated at the visit level and at the record level, considering approximately 10,000 individual codes and over a million bigram codes. Various aggregate statistics, such as (but not limited to) the number of visits per record, medical codes per visit, and prevalence of chronic disease labels are evaluated. The results are presented in FIG. 3, Table 2, and Table 4.

TABLE 2
Aggregate Statistics Regarding the Shape of Training and Compared Synthetic Datasets

| Outpatient EHR | | | Inpatient EHR | | |
|---|---|---|---|---|---|
| Record Length | → | Visit Length | Record Length | → | Visit Length |
| Mean(Std. Dev.) | → | Mean(Std. Dev.) | Mean(Std. Dev.) | → | Mean(Std. Dev.) |

| | | | | | | | | |
|---|---|---|---|---|---|---|---|---|
| EVA | 29.49 | (28.88) | 3.35 | (1.71) | 1.20 | (0.723) | 11.92 | (3.665) |
| SynTEG | 93.00 | (2.30) | 3.70 | (4.10) | 27.55 | (3.34) | 5.93 | (10.96) |
| LSTM | 32.04 | (27.14) | 3.22 | (1.64) | 1.30 | (0.56) | 9.53 | (2.91) |
| GPT | 95.72 | (3.37) | 2.70 | (1.73) | 1.26 | (0.73) | 9.67 | (5.45) |
| HALO-Coarse | 35.26 | (31.87) | 3.77 | (2.23) | 1.13 | (0.39) | 11.21 | (3.91) |
| HALO | 36.19 | (33.41) | 3.93 | (2.72) | 1.31 | (0.84) | 11.93 | (6.45) |
| Train Data | 34.18 | (32.35) | 3.52 | (2.18) | 1.27 | (0.92) | 11.68 | (5.70) |

Additionally, R² values for visit-level normalized code probabilities in our high-dimensional outpatient EHR dataset and a lower-dimensional setting are provided. The details can be found in Table 3.

TABLE 3
Code Probability Correlations R² between Training and Synthetic Datasets

| | High-Dimensional Outpatient EHR | | | Low-Dimensional Outpatient EHR | | |
|---|---|---|---|---|---|---|
| | | Sequential | | | Sequential | |
| | Unigram | Visit | Same Visit | Unigram | Visit | Same Visit |
| | Code | Bigram | Bigram | Code | Bigram | Bigram |
| | Probabilities | Probabilities | Probabilities | Probabilities | Probabilities | Probabilities |
| EVA | 0.910 | 0.082 | 0.128 | 0.957 | 0.134 | 0.225 |
| SynTEG | 0.915 | 0.355 | 0.082 | 0.784 | 0.315 | 0.211 |
| LSTM | 0.900 | 0.077 | 0.127 | 0.962 | 0.135 | 0.225 |
| GPT | 0.743 | 0.382 | 0.262 | 0.924 | 0.626 | .515 |
| HALO-Coarse | 0.794 | 0.357 | 0.176 | 0.882 | 0.503 | 0.247 |
| HALO | 0.914 | 0.508 | 0.362 | 0.949 | 0.686 | 0.562 |

Table 3 shows R² values that measure the correlations of the three types of code probabilities for different synthetic datasets against the training data in both high-dimensional and low-dimensional settings. Although the results showed a drop in performance for each method in the high-dimensional setting, the HALO platform 100 was able to maintain strong performance with minimal decline. Overall, the HALO platform 100 outperformed the baselines in both bigram evaluations in low and high dimensional settings.

FIGS. 3A-3C show code probability plots, illustrating Unigram, Sequential Visit Bigram, and Same Record Bigram probabilities for each synthetic dataset. With the exception of SynTEG, all models exhibit some correlation in the unigram and temporal bigram evaluations, but many have weak correlation or consistently yield higher synthetic probabilities due to a lack of temporal consistency and repetition across visits in the records. HALO and, to a lesser extent, HALO-Coarse perform the best in all settings. However, HALO is the only platform that realistically produced pairs of codes within and across visits.

The GPT baseline struggles with the complexity of the outpatient EHR dataset in terms of stopping the record generation. This is common to many language models in the text generation domain, as their overall quality decays for long sequences and the lack of visit level grouping in its data representation causes its sequences to be considerably longer. The language model architectures (GPT, LSTM, HALO-Coarse, and HALO) can model both the shape of the synthetic records and the temporal dependencies, on average, with higher accuracy than the VAE and GAN-based baselines. While each of the compared methods models the unigram code probabilities relatively well (As shown in FIGS. 3A-3C), the temporal modeling is better shown in the overall synthetic record and visit lengths, the generation of chronic disease labels, and the sequential bigram evaluation. SynTEG, EVA, and the LSTM baselines struggle with these evaluations (with the LSTM baseline struggling largely due simply to overall weakness).

The LSTM and HALO-Coarse model baselines falter with respect to same-visit bigram probabilities due to their lack of intra-visit dependency modeling. The GPT baseline, which models each code individually and so offers that intra-visit modeling, can maintain relatively stronger performance there. HALO model platform 100 combines and builds on the strengths of each baseline without any weaknesses, using the compact multi-hot representation to offer a powerful model that does not struggle with any length or feature of data while simultaneously maintaining the intra-visit modeling in an even more powerful and structured way. As such, the HALO platform 100 can best maintain performance in this high-dimensional setting. The HALO platform 100 produces improved results that closely model the true training data in all settings in terms of record and visit lengths, label probabilities, and all combinations of code probabilities. This signifies that HALO is capable of generating data that looks realistic.

A third evaluation explores the utility of synthetic datasets produced by the model 100 and the baselines for training disease classifiers. To this end, two different synthetically-supplemented data setups and machine learning classifiers may be used to predict chronic disease labels based on patient visits. In each of the two setups, a simple bidirectional LSTM with a single-layer fully connected head classifier is used to predict one or more chronic disease labels based on a set of patient visits.

In a first data setup, model performance is assessed in real-world scenarios using synthetic training data. Experiments may be conducted for each of the 11 chronic disease labels in the outpatient EHR dataset, sourced from (for example) the Centers for Medicare and Medicaid Services, and/or the SynPUF dataset. Additionally or alternatively, experiments may be conducted for each of the 25 chronic diseases in the inpatient EHR dataset.

For each chronic disease, 2,500 records may be randomly extracted for training, ensuring a balanced distribution of positive and negative labels (e.g., approximately 50-50). This process may be repeated across six synthetic datasets (one for each method) and one real training dataset, resulting in a total of seven balanced training datasets. The selected size of 2,500 records strikes a balance between having enough training data for machine learning models and maintaining sufficient positive labels for each disease, though those of skill in the art will recognize that more or fewer records may be extracted for training.

Classifiers may be trained on each of these datasets, and the classifiers may select the best model for each dataset using a validation set of, for example, 250 records, approximately equally representing both classes. The models may be evaluated on test sets containing a fixed number (e.g., 500) records, equally representing both classes, from the original test set comprising real patient data.

Table 4 shows average accuracy and F1 score for each synthetic dataset from each of the compared models, as well as the real training data across each of the chronic disease labels. The standard deviations of each metric are shown as well, but most of that deviation stems from differences between tasks rather than inconsistent performance within each model.

The average performance for each model is shown in terms of accuracy and F1 Score. The scores shown in Table 4 represent each of the 11 chronic disease labels in our outpatient dataset and 25 chronic disease labels in our inpatient dataset. The models were trained on each synthetic dataset and tested on real data. The reported values represent the mean and standard deviation across the tasks. GPT, HALO-Coarse, and HALO's data offer large improvements over the other baselines and perform similarly to real training data. The synthetic data of the HALO platform 100 performs the best, having the highest average performance of all synthetic methods.

TABLE 4
Chronic Disease Classification Model
Performance Trained on Synthetic Data

| | Outpatient EHR | | Inpatient EHR | |
|---|---|---|---|---|
| | Avg. | Avg. | Avg. | Avg. |
| | Accuracy | F1 Score | Accuracy | F1 Score |
| EVA | 0.508 ± 0.02 | 0.283 ± 0.26 | 0.5356 ± 0.05 | 0.580 ± 0.05 |
| SynTEG | 0.507 ± 0.03 | 0.514 ± 0.20 | 0.539 ± 0.06 | 0.438 ± 0.06 |
| LSTM | 0.506 ± 0.02 | 0.467 ± 0.28 | 0.522 ± 0.04 | 0.565 ± 0.04 |
| GPT | 0.851 ± 0.03 | 0.854 ± 0.03 | 0.877 ± 0.05 | 0.881 ± 0.05 |
| HALO- | 0.867 ± 0.03 | 0.863 ± 0.03 | 0.863 ± 0.05 | 0.865 ± 0.05 |
| Coarse | | | | |
| HALO | 0.879 ± 0.03 | 0.878 ± 0.03 | 0.882 ± 0.04 | 0.884 ± 0.04 |
| Real Data | 0.891 ± 0.03 | 0.895 ± 0.03 | 0.938 ± 0.04 | 0.937 ± 0.04 |

Additionally or alternatively, the utility of the synthetic EHR data may be evaluated in identifying uncommon conditions. A highly imbalanced dataset of patients labeled with cancer and/or other chronic disease may be formed from, for example, the outpatient EHR dataset. The imbalanced dataset included a fixed number (e.g., 50,000) of EHR records without the cancer chronic disease label and only 1,000 records with the label.

Using this imbalanced data, a classifier can be trained and performance thereof can be compared to classifiers trained on balanced datasets. For balancing, we added a number (e.g., 49,000) of positively labeled synthetic records to the imbalanced dataset to create a new, balanced dataset. Additionally or alternatively, the classifier may be trained on a dataset balanced using real records.

The evaluation results are summarized in Table 5. Notably, HALO outperformed all baselines, exhibiting significant improvements on the original unbalanced dataset as well as the synthetically augmented datasets. It approached the upper bound performance of the ideal balanced dataset. This experiment underscores the potential of synthetic EHR data in supporting the identification of uncommon conditions, and the additional benefits of the HALO platform 100.

TABLE 5
Rare Disease Detection Performance
on Synthetic Balanced Datasets

| | BCE Loss | Accuracy | F1 Score | AUROC |
|---|---|---|---|---|
| Original | 0.693 | 0.497 | 0.013 | 0.417 |
| imbalanced | | | | |
| Balanced with | 0.127 | 0.951 | 0.951 | 0.989 |
| real data | | | | |
| EVA | 0.615 | 0.695 | 0.705 | 0.730 |
| SynTEG | 0.598 | 0.735 | 0.758 | 0.786 |
| LSTM | 0.593 | 0.702 | 0.714 | 0.743 |
| GPT | 0.472 | 0.880 | 0.869 | 0.956 |
| HALO-Coarse | 0.265 | 0.918 | 0.916 | 0.959 |
| HALO | 0.192 | 0.931 | 0.931 | 0.976 |

As shown in Table 5, models trained on datasets balanced using each synthetic datasets can be compared against models trained on the original imbalanced data (representing the rare disease dataset) and/or against an upper-bound ideal dataset balanced using real data. Among the evaluated models, EVA and SynTEG exhibit limited utility, while the language model architectures LSTM, GPT, and HALO-Coarse offer substantial value. The HALO platform 100 leading performance, closely approaching the results of a true, balanced dataset.

### Realistic Continuous Variables in Synthetic EHRS

The viability of the discretized representation of continuous values in the HALO platform 100, and the effectiveness of using it to model those variables. One or more new training datasets including visit gaps may be built using the outpatient EHR dataset and/or the inpatient EHR dataset. The one or more datasets may be used to train a new version of the HALO model 100 and generate a synthetic dataset including 250,000 variables and 45,000 records.

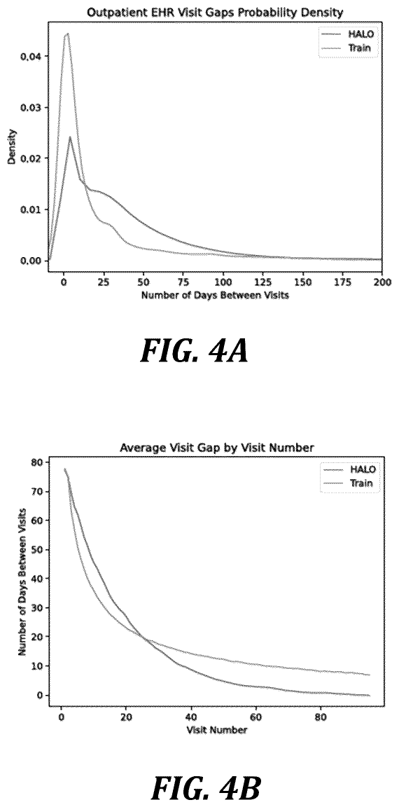

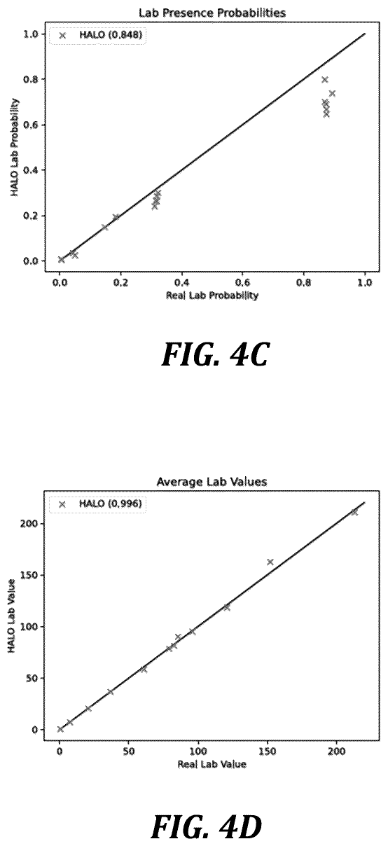

FIGS. 4A-4D show how various distributions of the variables from the synthetic dataset match the real values. The HALO platform 100 effectively captures the distribution of continuous variables through its discretization approach, as demonstrated in four scenarios: inter-visit gap probability density, inter-visit gap by visit number, lab presence probabilities, and mean lab values.

As shown in FIG. 4A, the intervisit gap probability density represents the probability density of inter-visit gaps. The HALO platform 100 generates a synthetic EHR that closely approximates the true shape of real data. FIG. 4B shows inter-visit gap by visit number: The mean visit gap, as per visit number, across both real and synthetic datasets reveals that HALO accurately captures the pattern of patients with many records, showing shorter gaps in their subsequent visits. Thus, as shown in FIGS. 4A and 4B, the HALO platform 100 accurately replicates the distribution of gaps between patient visits and the pattern of shorter gaps for longer records, respectively. These captured nuanced patterns are on top of the aggregate mean gaps being very similar as well. In particular, there are 33.53 days between visits on average within the real outpatient EHR data and 35.77 days on average for the synthetic data generated by the HALO platform 100.

FIG. 4C shows a comparison of lab presence probabilities between the synthetic data produced by the HALO platform 100 and the real EHR data. The probability of binary lab presence demonstrates that the HALO platform 100 accurately generates lab variables, even when discretized across multiple variables. FIG. 4D shows a comparison of mean lab values between the synthetic data produced by the HALO platform 100 and the real EHR data. The average value of labs, when present, confirms that the synthetic labs produced using the HALO platform 100 closely resemble those of the real dataset.

Thus, the HALO platform 100 replicates not only the presence (see FIG. 4C) of lab values, but also the average values (see FIG. 4D) of performed lab tests. Overall, the discretization of continuous variables performed by the HALO platform 100 is effective. The platform 100 has the potential to generate comprehensive synthetic patient records with multiple variables of different types.

### Privacy Evaluation of Synthetic EHRS

In addition to the high fidelity of synthetic EHRs generated by the HALO platform 100, the platform also seeks to protect the privacy of the patients within the original training dataset. To that end, a dataset produced by the platform 100 was exposed to a commonly used membership inference attack to test identification risk for the dataset.

One evaluation of the dataset is the ability to thwart a membership inference attack. A membership inference attack aims to determine whether any specific real patient record was used in the training dataset to generate the synthetic records. Membership inference attacks are a well-known privacy test in the field of synthetic EHR generation, and addressing them is important to help ensure the privacy and confidentiality of patient identities.

To demonstrate that the HALO platform 100 produces data that is not susceptible to such an attack, two different attempts at a membership inference attack were performed, one based on the synthetic data generator, and another based on the synthetic dataset itself. To generate an attack dataset, a number of records (e.g., 100,000) may be selected from one or more (e.g., each) real dataset used for training. The selected datasets may be assigned a positive label. A similar number (e.g., 100,000) records may be selected from among the remaining records not used for training and assigned a negative label.

Two attacks may be conducted using the attack dataset:
In a Model Attack, a number (e.g., 100,000) of records with the highest log probability from the model are labeled as positive, predicting that they were part of the training dataset.
In a Dataset Attack, a number (e.g., 100,000) of records with the lowest hamming distance to the closest record in the synthetic dataset are labeled as positive. A hamming distance (equivalent to Manhattan Distance in a binary setting) is selected as a distance metric between patient records throughout privacy evaluations. Alternatively, any other distance metric could be substituted interchangeably.

These two attacks allow us to test the ability of the synthetic dataset to prevent an attacker from inferring whether a real record was used in the training dataset.

The results of the classifications from the attacks are shown in Table 6. The accuracy of both attacks on both datasets is approximately 50%, which is similar to a random guess. This shows that neither the model nor the synthetic dataset reveals any meaningful or compromising information about the patient identity in the training dataset. The dataset attack was also performed with each of the baseline datasets. Table 6 shows that each baseline similarly accomplishes the goal of privacy, achieving a similar probability at around 50%. The model attack was not performed on the baselines because most of the baselines cannot offer a probability output of input patient records.

TABLE 6
Membership Inference Attack Results

| | Outpatient EHR | | | Inpatient EHR | | |
|---|---|---|---|---|---|---|
| | Acc. | Precision | Recall | Acc. | Precision | Recall |
| HALO Dataset Attack | 0.51 | 0.501 | 0.501 | 03492 | 0.491 | 0.477 |
| HALO Model Attack | 0.509 | 0.509 | 0.509 | 0.515 | 0.515 | 0.515 |
| EVA Dataset Attack | 0.498 | 0.498 | 0.496 | 0.493 | 0.493 | 0.477 |
| SynTEG Dataset Attack | 0.500 | 0.500 | 0.500 | 0.491 | 0.491 | 0.467 |
| LSTM Dataset Attack | 0.499 | 0.499 | 0.496 | 0.494 | 0.494 | 0.481 |
| GPT Dataset Attack | 0.500 | 0.500 | 0.500 | 0.492 | 0.491 | 0.455 |
| HALO-Coarse Dataset | 0.500 | 0.500 | 0.499 | 0.491 | 0.491 | 0.462 |
| Attack | | | | | | |

As shown in Table 6. for each record in the attack dataset, both the log probability of the record from the trained model (Model Attack) is found for the HALO platform, and the hamming distance to the closest record in the synthetic dataset (Dataset Attack) are found for the HALO platform and the baseline platforms. The attacks label the half of the records with the highest probability or lowest distance records, respectively, as in the training set. The accuracy for either attack is around 50%, which is similar to a random guess. This indicates that the synthetic dataset and the model do not reveal any patient-identifying information about the original training datasets. Each baseline synthetic dataset similarly thwarts the dataset attack.

The HALO platform 100 generates high-dimensional synthetic longitudinal EHR data. In particular, the method performed by the HALO platform 100 is specifically designed to handle the sequential, multi-granular, and high-dimensional nature of EHR data by generating an explicit probability distribution over the codes, visits, and records. The HALO platform 100 can generate realistic data without needing to aggregate or remove any codes, as conventional approaches have unanimously done. Moreover, the HALO platform 100 can produce realistic synthetic EHR data. Specifically, HALO can capture the probability distribution underlying the records better than other language model baselines, and can produce a synthetic dataset that both looks similar to and offers the utility of real patient records, as measured by medical code occurrence probabilities and machine learning classification tasks augmented with synthetic data. Still further, the HALO platform 100 helps to protect privacy of the underlying EHR data used as training data.

### Methods of Use

Embodiments of the present disclosure provide a hardware and software platform operative by a set of methods and computer-readable media comprising instructions configured to operate the aforementioned modules and computing elements in accordance with the methods. The following depicts an example of at least one method of a plurality of methods that may be performed by at least one of the aforementioned modules. Various hardware components may be used at the various stages of operations disclosed with reference to each module.

For example, although methods may be described as being performed by a single computing device, it should be understood that, in some embodiments, different operations may be performed by different networked elements in operative communication with the computing device. For example, at least one computing device 600 may be employed in the performance of some or all of the stages disclosed with regard to the methods. Similarly, an apparatus may be employed in the performance of some or all of the stages of the methods. As such, the apparatus may comprise at least those architectural components found in computing device 600.

Furthermore, although the stages of the following example method are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in arrangements that differ from the ones described below. Moreover, various stages may be added or removed from the without altering or departing from the fundamental scope of the depicted methods and systems disclosed herein.

A. Master Method

Consistent with embodiments of the present disclosure, a method may be performed by at least one of the aforementioned modules. The method may be embodied as, for example, but not limited to, computer instructions, which, when executed, perform the method.

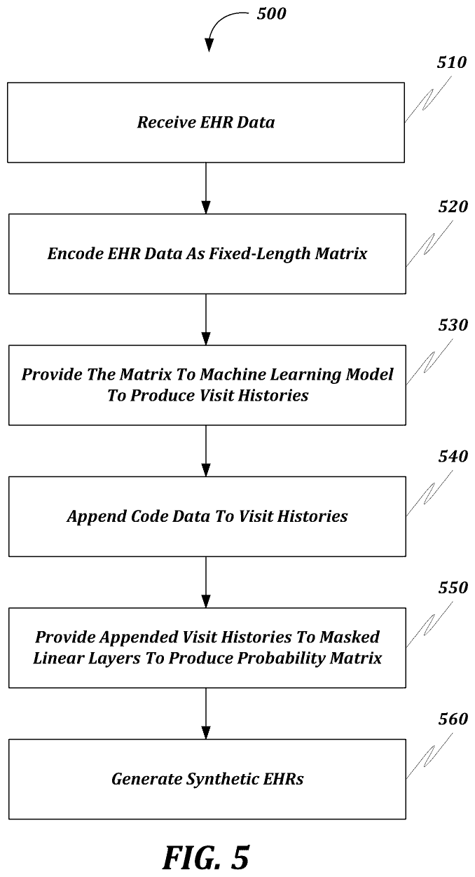

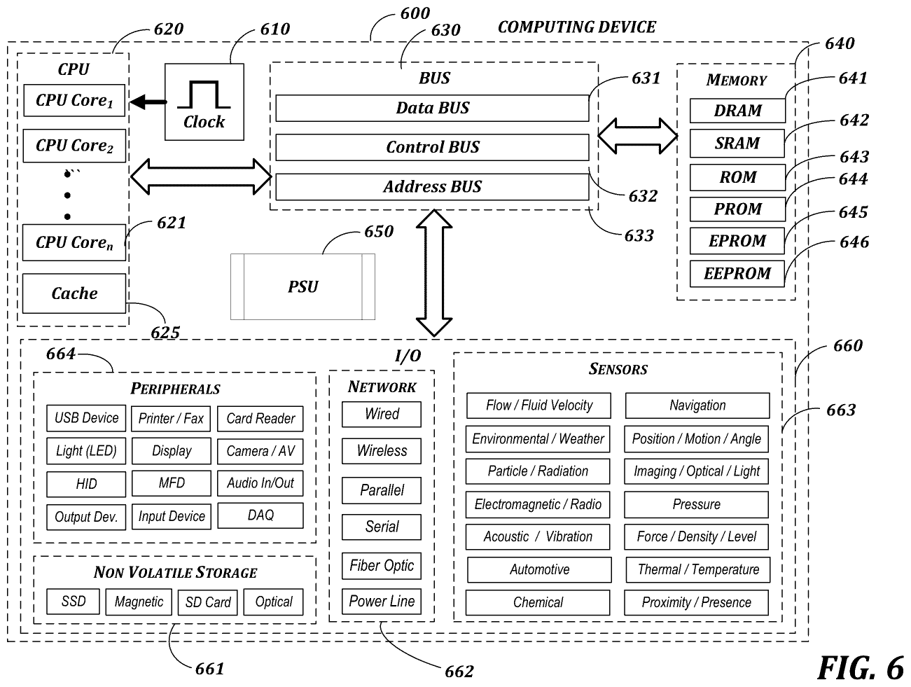

FIG. 5 is a flow chart setting forth the general stages involved in a method 500 consistent with an embodiment of the disclosure for providing the HALO platform 100. Method 500 may be implemented using a computing device 600 or any other component associated with platform 100 as described in more detail below with respect to FIG. 6. For illustrative purposes alone, computing device 600 is described as one potential actor in the following stages.

Method 500 may begin at stage 510 where computing device 600 may Receive EHR data. For example, the EHR data may be provided as a data file or other large data store. In embodiments, the EHR data may be converted to a matrix representation. As a particular example, the matrix representation may have a separate column for each visit, and may represent each code variable as a separate row. In some embodiments, the columns may include a first column comprising a start visit that indicates a beginning of the EHR, a second column comprising a label visit that stores one or more labels associated with demographic information and/or chronic disease information associated with the patient, one or more visit columns that store medical codes associated with a patient visit, an end column that represents a final visit, and/or one or more padding columns for use in shaping the matrix. In some embodiments, the rows may include a first set of rows representing medical codes, a second set of rows representing label codes, one or more rows representing a star code, one or more rows representing an end code, one or more rows representing a time between visits, and/or one or more rows representing data padding for use in shaping the matrix.

From stage 510, where computing device 600 receives EHR data, method 500 may advance to stage 520 where computing device 600 may encode the received EHR data into a fixed-length vector. For example, the EHR data may be provided to one or more embedding layers to format the data. As examples, the EHR data may be provided to a code embedding matrix that maps each visit code to a dense vector representation and/or a positional embedding matrix that captures the relative position of each visit in sequence.

Once computing device 600 converts the EHR data to a fixed length vector in stage 520, method 500 may continue to stage 530 where computing device 600 may provide the vector as input to a machine learning model. In particular, the machine learning model may comprise one or more transformer modules. For example a stack of a plurality (e.g., 12) of transformer modules may be used as the machine learning model. The machine learning model may output a sequence of visit history representations that capture the collective information of all previous visits.

After computing device 600 creates the visit history outputs in stage 530, method 500 may proceed to stage 540 where computing device 600 may append code information to one or more (e.g., each) visit in the visit history output to create an appended visit history. For example, the code data may be appended such that each visit history record in the appended visit history includes the history of all previous visits and the codes of the current visit, so that each input has a representation of the history of all the previous visits and the codes of the current visit.

After creating the appended visit history in stage 540, method 500 may proceed to stage 550 where the computing device 600 may provide the appended visit history to one or more masked linear layers to model the probability distribution for each variable in each visit of the EHR. The one or more masked linear layers may use an upper triangular masking to preserve the autoregressive property of the probabilities. In embodiments, the one or more masked linear layers may maintain the same dimensionality of the appended visit history. The output from the one or more masked linear layers may be a matrix of probabilities of each code for each visit after the start visit built from the visit histories and each previous code in the same visit. In some embodiments, the computing device 600 may normalize the probability matrix by applying one or more normalization functions thereto. Thereafter, at stage 560, the computing device may generate (synthesize) a set of synthetic electronic health records based on the output probability matrix. The synthetic EHRs may be used to train one or more further machine learning models.

### Hardware Configuration

Embodiments of the present disclosure provide a hardware and software platform operative as a distributed system of modules and computing elements.

Platform 100 may be embodied as, for example, but not be limited to, a website, a web application, a desktop application, a backend application, and a mobile application compatible with a computing device 600. The computing device 600 may comprise, but not be limited to, the following:
Mobile computing device, such as, but is not limited to, a laptop, a tablet, a smartphone, a drone, a wearable, an embedded device, a handheld device, an Arduino, an industrial device, or a remotely operable recording device;
A supercomputer, an exascale supercomputer, a mainframe, or a quantum computer;
A minicomputer, wherein the minicomputer computing device comprises, but is not limited to, an IBM AS400/iSeries/System I, A DEC VAX/PDP, an HP3000, a Honeywell-Bull DPS, a Texas Instruments TI-990, or a Wang Laboratories VS Series;
A microcomputer, wherein the microcomputer computing device comprises, but is not limited to, a server, wherein a server may be rack-mounted, a workstation, an industrial device, a raspberry pi, a desktop, or an embedded device;
Platform 100 may be hosted on a centralized server or a cloud computing service. Although method 500 has been described to be performed by a computing device 600, it should be understood that, in some embodiments, different operations may be performed by a plurality of the computing devices 600 in operative communication on at least one network.

Embodiments of the present disclosure may comprise a system having a central processing unit (CPU) 620, a bus 630, a memory unit 640, a power supply unit (PSU) 650, and one or more Input/Output (I/O) units. The CPU 620 coupled to the memory unit 640 and the plurality of I/O units 660 via the bus 630, all of which are powered by the PSU 650. It should be understood that, in some embodiments, each disclosed unit may actually be a plurality of such units for redundancy, high availability, and/or performance purposes. The combination of the presently disclosed units is configured to perform the stages of any method disclosed herein.

FIG. 6 is a block diagram of a system including computing device 600. Consistent with an embodiment of the disclosure, the aforementioned CPU 620, the bus 630, the memory unit 640, a PSU 650, and the plurality of I/O units 660 may be implemented in a computing device, such as computing device 600 of FIG. 6. Any suitable combination of hardware, software, or firmware may be used to implement the aforementioned units. For example, the CPU 620, the bus 630, and the memory unit 640 may be implemented with computing device 600 or any of other computing devices 600, in combination with computing device 600. The aforementioned system, device, and components are examples and other systems, devices, and components may comprise the aforementioned CPU 620, the bus 630, and the memory unit 640, consistent with embodiments of the disclosure.

At least one computing device 600 may be embodied as any of the computing elements illustrated in all of the attached figures. A computing device 600 does not need to be electronic, nor even have a CPU 620, nor bus 630, nor memory unit 640. The definition of the computing device 600 to a person having ordinary skill in the art is “A device that computes, especially a programmable [usually] electronic machine that performs high-speed mathematical or logical operations or that assembles, stores, correlates, or otherwise processes information.” Any device which processes information qualifies as a computing device 600, especially if the processing is purposeful.

With reference to FIG. 6, a system consistent with an embodiment of the disclosure may include a computing device, such as computing device 600. In some configurations, the computing device 600 may include at least one clock module 610, at least one CPU 620, at least one bus 630, and at least one memory unit 640, at least one PSU 650, and at least one I/O 660 module, wherein I/O module may be comprised of, but not limited to a non-volatile storage sub-module 661, a communication sub-module 662, a sensors sub-module 663, and a peripherals sub-module 664.

In a system consistent with an embodiment of the disclosure, the computing device 600 may include the clock module 610, known to a person having ordinary skill in the art as a clock generator, which produces clock signals. Clock signals may oscillate between a high state and a low state at a controllable rate, and may be used to synchronize or coordinate actions of digital circuits. Most integrated circuits (ICs) of sufficient complexity use a clock signal in order to synchronize different parts of the circuit, cycling at a rate slower than the worst-case internal propagation delays. One well-known example of the aforementioned integrated circuit is the CPU 620, the central component of modern computers, which relies on a clock signal. The clock 610 can comprise a plurality of embodiments, such as, but not limited to, a single-phase clock which transmits all clock signals on effectively 1 wire, a two-phase clock which distributes clock signals on two wires, each with non-overlapping pulses, and a four-phase clock which distributes clock signals on 4 wires.

Many computing devices 600 may use a “clock multiplier” which multiplies a lower frequency external clock to the appropriate clock rate of the CPU 620. This allows the CPU 620 to operate at a much higher frequency than the rest of the computing device 600, which affords performance gains in situations where the CPU 620 does not need to wait on an external factor (like memory 640 or input/output 660). Some embodiments of the clock 610 may include dynamic frequency change, where, the time between clock edges can vary widely from one edge to the next and back again.

In a system consistent with an embodiment of the disclosure, the computing device 600 may include the CPU 620 comprising at least one CPU Core 621. In other embodiments, the CPU 620 may include a plurality of identical CPU cores 621, such as, but not limited to, homogeneous multi-core systems. It is also possible for the plurality of CPU cores 621 to comprise different CPU cores 621, such as, but not limited to, heterogeneous multi-core systems, big.LITTLE systems and some AMD accelerated processing units (APU). The CPU 620 reads and executes program instructions which may be used across many application domains, for example, but not limited to, general purpose computing, embedded computing, network computing, digital signal processing (DSP), and graphics processing (GPU). The CPU 620 may run multiple instructions on separate CPU cores 621 simultaneously. The CPU 620 may be integrated into at least one of a single integrated circuit die, and multiple dies in a single chip package. The single integrated circuit die and/or the multiple dies in a single chip package may contain a plurality of other elements of the computing device 600, for example, but not limited to, the clock 610, the bus 630, the memory 640, and I/O 660.

The CPU 620 may contain cache 622 such as but not limited to a level 1 cache, a level 2 cache, a level 3 cache, or combinations thereof. The cache 622 may or may not be shared amongst a plurality of CPU cores 621. The cache 622 sharing may comprise at least one of message passing and inter-core communication methods used for the at least one CPU Core 621 to communicate with the cache 622. The inter-core communication methods may comprise, but not be limited to, bus, ring, two-dimensional mesh, and crossbar. The aforementioned CPU 620 may employ symmetric multiprocessing (SMP) design.

The one or more CPU cores 621 may comprise soft microprocessor cores on a single field programmable gate array (FPGA), such as semiconductor intellectual property cores (IP Core). The architectures of the one or more CPU cores 621 may be based on at least one of, but not limited to, Complex Instruction Set Computing (CISC), Zero Instruction Set Computing (ZISC), and Reduced Instruction Set Computing (RISC). At least one performance-enhancing method may be employed by one or more of the CPU cores 621, for example, but not limited to Instruction-level parallelism (ILP) such as, but not limited to, superscalar pipelining, and Thread-level parallelism (TLP).

Consistent with the embodiments of the present disclosure, the aforementioned computing device 600 may employ a communication system that transfers data between components inside the computing device 600, and/or the plurality of computing devices 600. The aforementioned communication system will be known to a person having ordinary skill in the art as a bus 630. The bus 630 may embody internal and/or external hardware and software components, for example, but not limited to a wire, an optical fiber, various communication protocols, and/or any physical arrangement that provides the same logical function as a parallel electrical bus. The bus 630 may comprise at least one of a parallel bus, wherein the parallel bus carries data words in parallel on multiple wires; and a serial bus, wherein the serial bus carries data in bit-wise serial form. The bus 630 may embody a plurality of topologies, for example, but not limited to, a multidrop/electrical parallel topology, a daisy chain topology, and connected by switched hubs, such as a USB bus. The bus 630 may comprise a plurality of embodiments, for example, but not limited to:
Internal data bus (data bus) 631/Memory bus
Control bus 632
Address bus 633
System Management Bus (SMBus)
Front-Side-Bus (FSB)
External Bus Interface (EBI)
Local bus
Expansion bus
Lightning bus
Controller Area Network (CAN bus)
Camera Link
ExpressCard
Advanced Technology management Attachment (ATA), including embodiments and derivatives such as, but not limited to, Integrated Drive Electronics (IDE)/Enhanced IDE (EIDE), ATA Packet Interface (ATAPI), Ultra-Direct Memory Access (UDMA), Ultra ATA (UATA)/Parallel ATA (PATA)/Serial ATA (SATA), CompactFlash (CF) interface, Consumer Electronics ATA (CE-ATA)/Fiber Attached Technology Adapted (FATA), Advanced Host Controller Interface (AHCI), SATA Express (SATAe)/External SATA (eSATA), including the powered embodiment eSATAp/Mini-SATA (mSATA), and Next Generation Form Factor (NGFF)/M.2.

Small Computer System Interface (SCSI)/Serial Attached SCSI (SAS)
HyperTransport
InfiniBand
RapidIO
Mobile Industry Processor Interface (MIPI)
Coherent Processor Interface (CAPI)
Plug-n-play
1-Wire
Peripheral Component Interconnect (PCI), including embodiments such as but not limited to, Accelerated Graphics Port (AGP), Peripheral Component Interconnect eXtended (PCI-X), Peripheral Component Interconnect Express (PCI-e) (e.g., PCI Express Mini Card, PCI Express M.2 [Mini PCIe v 2], PCI Express External Cabling [ePCIe], and PCI Express OCuLink [Optical Copper{Cu} Link]), Express Card, AdvancedTCA, AMC, Universal IO, Thunderbolt/Mini DisplayPort, Mobile PCIe (M-PCIe), U.2, and Non-Volatile Memory Express (NVMe)/Non-Volatile Memory Host Controller Interface Specification (NVMHCIS).
Industry Standard Architecture (ISA), including embodiments such as, but not limited to Extended ISA (EISA), PC/XT-bus/PC/AT-bus/PC/104 bus (e.g., PC/104-Plus, PCI/104-Express, PCI/104, and PCI-104), and Low Pin Count (LPC).
Music Instrument Digital Interface (MIDI)
Universal Serial Bus (USB), including embodiments such as, but not limited to, Media Transfer Protocol (MTP)/Mobile High-Definition Link (MHL), Device Firmware Upgrade (DFU), wireless USB, InterChip USB, IEEE 1394 Interface/Firewire, Thunderbolt, and eXtensible Host Controller Interface (xHCI).

Consistent with the embodiments of the present disclosure, the aforementioned computing device 600 may employ hardware integrated circuits that store information for immediate use in the computing device 600, known to persons having ordinary skill in the art as primary storage or memory 640. The memory 640 operates at high speed, distinguishing it from the non-volatile storage sub-module 661, which may be referred to as secondary or tertiary storage, which provides relatively slower-access to information but offers higher storage capacity. The data contained in memory 640, may be transferred to secondary storage via techniques such as, but not limited to, virtual memory and swap. The memory 640 may be associated with addressable semiconductor memory, such as integrated circuits consisting of silicon-based transistors, that may be used as primary storage or for other purposes in the computing device 600. The memory 640 may comprise a plurality of embodiments, such as, but not limited to volatile memory, non-volatile memory, and semi-volatile memory. It should be understood by a person having ordinary skill in the art that the following are non-limiting examples of the aforementioned memory:
Volatile memory, which requires power to maintain stored information, for example, but not limited to, Dynamic Random-Access Memory (DRAM) 641, Static Random-Access Memory (SRAM) 642, CPU Cache memory 625, Advanced Random-Access Memory (A-RAM), and other types of primary storage such as Random-Access Memory (RAM).
Non-volatile memory, which can retain stored information even after power is removed, for example, but not limited to, Read-Only Memory (ROM) 643, Programmable ROM (PROM) 644, Erasable PROM (EPROM) 645, Electrically Erasable PROM (EEPROM) 646 (e.g., flash memory and Electrically Alterable PROM [EAPROM]), Mask ROM (MROM), One Time Programmable (OTP) ROM/Write Once Read Many (WORM), Ferroelectric RAM (FeRAM), Parallel Random-Access Machine (PRAM), Split-Transfer Torque RAM (STT-RAM), Silicon Oxime Nitride Oxide Silicon (SONOS), Resistive RAM (RRAM), Nano RAM (NRAM), 3D XPoint, Domain-Wall Memory (DWM), and millipede memory.
Semi-volatile memory may have limited non-volatile duration after power is removed but may lose data after said duration has passed. Semi-volatile memory provides high performance, durability, and other valuable characteristics typically associated with volatile memory, while providing some benefits of true non-volatile memory. The semi-volatile memory may comprise volatile and non-volatile memory, and/or volatile memory with a battery to provide power after power is removed. The semi-volatile memory may comprise, but is not limited to, spin-transfer torque RAM (STT-RAM).

Consistent with the embodiments of the present disclosure, the aforementioned computing device 600 may employ a communication system between an information processing system, such as the computing device 600, and the outside world, for example, but not limited to, human, environment, and another computing device 600. The aforementioned communication system may be known to a person having ordinary skill in the art as an Input/Output (I/O) module 660. The I/O module 660 regulates a plurality of inputs and outputs with regard to the computing device 600, wherein the inputs are a plurality of signals and data received by the computing device 600, and the outputs are the plurality of signals and data sent from the computing device 600. The I/O module 660 interfaces with a plurality of hardware, such as, but not limited to, non-volatile storage 661, communication devices 662, sensors 663, and peripherals 664. The plurality of hardware is used by at least one of, but not limited to, humans, the environment, and another computing device 600 to communicate with the present computing device 600. The I/O module 660 may comprise a plurality of forms, for example, but not limited to channel I/O, port mapped I/O, asynchronous I/O, and Direct Memory Access (DMA).

Consistent with the embodiments of the present disclosure, the aforementioned computing device 600 may employ a non-volatile storage sub-module 661, which may be referred to by a person having ordinary skill in the art as one of secondary storage, external memory, tertiary storage, off-line storage, and auxiliary storage. The non-volatile storage sub-module 661 may not be accessed directly by the CPU 620 without using an intermediate area in the memory 640. The non-volatile storage sub-module 661 may not lose data when power is removed and may be orders of magnitude less costly than storage used in memory 640. Further, the non-volatile storage sub-module 661 may have a slower speed and higher latency than in other areas of the computing device 600. The non-volatile storage sub-module 661 may comprise a plurality of forms, such as, but not limited to, Direct Attached Storage (DAS), Network Attached Storage (NAS), Storage Area Network (SAN), nearline storage, Massive Array of Idle Disks (MAID), Redundant Array of Independent Disks (RAID), device mirroring, off-line storage, and robotic storage. The non-volatile storage sub-module (661) may comprise a plurality of embodiments, such as, but not limited to:
Optical storage, for example, but not limited to, Compact Disk (CD) (CD-ROM/CD-R/CD-RW), Digital Versatile Disk (DVD) (DVD-ROM/DVD-R/DVD+R/DVD-RW/DVD+RW/DVD±RW/DVD+R DL/DVD-RAM/HD-DVD), Blu-ray Disk (BD) (BD-ROM/BD-R/BD-RE/BD-R DL/BD-RE DL), and Ultra-Density Optical (UDO).
Semiconductor storage, for example, but not limited to, flash memory, such as, but not limited to, USB flash drive, Memory card, Subscriber Identity Module (SIM) card, Secure Digital (SD) card, Smart Card, CompactFlash (CF) card, Solid-State Drive (SSD) and memristor.
Magnetic storage such as, but not limited to, Hard Disk Drive (HDD), tape drive, carousel memory, and Card Random-Access Memory (CRAM).
Phase-change memory
Holographic data storage such as Holographic Versatile Disk (HVD).
Molecular Memory
Deoxyribonucleic Acid (DNA) digital data storage

Consistent with the embodiments of the present disclosure, the computing device 600 may employ a communication sub-module 662 as a subset of the I/O module 660, which may be referred to by a person having ordinary skill in the art as at least one of, but not limited to, a computer network, a data network, and a network. The network may allow computing devices 600 to exchange data using connections, which may also be known to a person having ordinary skill in the art as data links, which may include data links between network nodes. The nodes may comprise networked computer devices 600 that may be configured to originate, route, and/or terminate data. The nodes may be identified by network addresses and may include a plurality of hosts consistent with the embodiments of a computing device 600. Examples of computing devices that may include a communication sub-module 662 include, but are not limited to, personal computers, phones, servers, drones, and networking devices such as, but not limited to, hubs, switches, routers, modems, and firewalls.

Two nodes can be considered networked together when one computing device 600 can exchange information with the other computing device 600, regardless of any direct connection between the two computing devices 600. The communication sub-module 662 supports a plurality of applications and services, such as, but not limited to World Wide Web (WWW), digital video and audio, shared use of application and storage computing devices 600, printers/scanners/fax machines, email/online chat/instant messaging, remote control, distributed computing, etc. The network may comprise one or more transmission mediums, such as, but not limited to conductive wire, fiber optics, and wireless signals. The network may comprise one or more communications protocols to organize network traffic, wherein application-specific communications protocols may be layered, and may be known to a person having ordinary skill in the art as being improved for carrying a specific type of payload, when compared with other more general communications protocols. The plurality of communications protocols may comprise, but are not limited to, IEEE 802, ethernet, Wireless LAN (WLAN/Wi-Fi), Internet Protocol (IP) suite (e.g., TCP/IP, UDP, Internet Protocol version 4 [IPv4], and Internet Protocol version 6 [IPv6]), Synchronous Optical Networking (SONET)/Synchronous Digital Hierarchy (SDH), Asynchronous Transfer Mode (ATM), and cellular standards (e.g., Global System for Mobile Communications [GSM], General Packet Radio Service [GPRS], Code-Division Multiple Access [CDMA], Integrated Digital Enhanced Network [IDEN], Long Term Evolution [LTE], LTE-Advanced [LTE-A], and fifth generation [5G] communication protocols).

The communication sub-module 662 may comprise a plurality of size, topology, traffic control mechanisms and organizational intent policies. The communication sub-module 662 may comprise a plurality of embodiments, such as, but not limited to:
Wired communications, such as, but not limited to, coaxial cable, phone lines, twisted pair cables (ethernet), and InfiniBand.
Wireless communications, such as, but not limited to, communications satellites, cellular systems, radio frequency/spread spectrum technologies, IEEE 802.11 Wi-Fi, Bluetooth, NFC, free-space optical communications, terrestrial microwave, and Infrared (IR) communications. Wherein cellular systems embody technologies such as, but not limited to, 3G, 4G (such as WiMAX and LTE), and 5G (short and long wavelength).
Parallel communications, such as, but not limited to, LPT ports.
Serial communications, such as, but not limited to, RS-232 and USB.
Fiber Optic communications, such as, but not limited to, Single-mode optical fiber (SMF) and Multi-mode optical fiber (MMF).
Power Line communications

The aforementioned network may comprise a plurality of layouts, such as, but not limited to, bus networks such as Ethernet, star networks such as Wi-Fi, ring networks, mesh networks, fully connected networks, and tree networks. The network can be characterized by its physical capacity or its organizational purpose. Use of the network, including user authorization and access rights, may differ according to the layout of the network. The characterization may include, but is not limited to a nanoscale network, a Personal Area Network (PAN), a Local Area Network (LAN), a Home Area Network (HAN), a Storage Area Network (SAN), a Campus Area Network (CAN), a backbone network, a Metropolitan Area Network (MAN), a Wide Area Network (WAN), an enterprise private network, a Virtual Private Network (VPN), and a Global Area Network (GAN).

Consistent with the embodiments of the present disclosure, the aforementioned computing device 600 may employ a sensors sub-module 663 as a subset of the I/O 660. The sensors sub-module 663 comprises at least one of the device, module, or subsystem whose purpose is to detect events or changes in its environment and send the information to the computing device 600. Sensors may be sensitive to the property they are configured to measure, may not be sensitive to any property not measured but be encountered in its application, and may not significantly influence the measured property. The sensors sub-module 663 may comprise a plurality of digital devices and analog devices, wherein if an analog device is used, an Analog to Digital (A-to-D) converter must be employed to interface the said device with the computing device 600. The sensors may be subject to a plurality of deviations that limit sensor accuracy. The sensors sub-module 663 may comprise a plurality of embodiments, such as, but not limited to, chemical sensors, automotive sensors, acoustic/sound/vibration sensors, electric current/electric potential/magnetic/radio sensors, environmental/weather/moisture/humidity sensors, flow/fluid velocity sensors, ionizing radiation/particle sensors, navigation sensors, position/angle/displacement/distance/speed/acceleration sensors, imaging/optical/light sensors, pressure sensors, force/density/level sensors, thermal/temperature sensors, and proximity/presence sensors. It should be understood by a person having ordinary skill in the art that the ensuing are non-limiting examples of the aforementioned sensors:
Chemical sensors, such as, but not limited to, breathalyzer, carbon dioxide sensor, carbon monoxide/smoke detector, catalytic bead sensor, chemical field-effect transistor, chemiresistor, electrochemical gas sensor, electronic nose, electrolyte-insulator-semiconductor sensor, energy-dispersive X-ray spectroscopy, fluorescent chloride sensors, holographic sensor, hydrocarbon dew point analyzer, hydrogen sensor, hydrogen sulfide sensor, infrared point sensor, ion-selective electrode, nondispersive infrared sensor, microwave chemistry sensor, nitrogen oxide sensor, olfactometer, optode, oxygen sensor, ozone monitor, pellistor, pH glass electrode, potentiometric sensor, redox electrode, zinc oxide nanorod sensor, and biosensors (such as nanosensors).

Automotive sensors, such as, but not limited to, air flow meter/mass airflow sensor, air-fuel ratio meter, AFR sensor, blind spot monitor, engine coolant/exhaust gas/cylinder head/transmission fluid temperature sensor, hall effect sensor, wheel/automatic transmission/turbine/vehicle speed sensor, airbag sensors, brake fluid/engine crankcase/fuel/oil/tire pressure sensor, camshaft/crankshaft/throttle position sensor, fuel/oil level sensor, knock sensor, light sensor, MAP sensor, oxygen sensor (o2), parking sensor, radar sensor, torque sensor, variable reluctance sensor, and water-in-fuel sensor.
Acoustic, sound and vibration sensors, such as, but not limited to, microphone, lace sensors such as a guitar pickup, seismometer, sound locator, geophone, and hydrophone.
Electric current, electric potential, magnetic, and radio sensors, such as, but not limited to, current sensor, Daly detector, electroscope, electron multiplier, faraday cup, galvanometer, hall effect sensor, hall probe, magnetic anomaly detector, magnetometer, magnetoresistance, MEMS magnetic field sensor, metal detector, planar hall sensor, radio direction finder, and voltage detector.
Environmental, weather, moisture, and humidity sensors, such as, but not limited to, actinometer, air pollution sensor, moisture alarm, ceilometer, dew warning, electrochemical gas sensor, fish counter, frequency domain sensor, gas detector, hook gauge evaporimeter, humistor, hygrometer, leaf sensor, lysimeter, pyranometer, pyrgeometer, psychrometer, rain gauge, rain sensor, seismometers, SNOTEL, snow gauge, soil moisture sensor, stream gauge, and tide gauge.
Flow and fluid velocity sensors, such as, but not limited to, air flow meter, anemometer, flow sensor, gas meter, mass flow sensor, and water meter.
Ionizing radiation and particle sensors, such as, but not limited to, cloud chamber, Geiger counter, Geiger-Muller tube, ionization chamber, neutron detection, proportional counter, scintillation counter, semiconductor detector, and thermoluminescent dosimeter.
Navigation sensors, such as, but not limited to, airspeed indicator, altimeter, attitude indicator, depth gauge, fluxgate compass, gyroscope, inertial navigation system, inertial reference unit, magnetic compass, MHD sensor, ring laser gyroscope, turn coordinator, variometer, vibrating structure gyroscope, and yaw rate sensor.
Position, angle, displacement, distance, speed, and acceleration sensors, such as but not limited to, accelerometer, displacement sensor, flex sensor, free-fall sensor, gravimeter, impact sensor, laser rangefinder, LIDAR, odometer, photoelectric sensor, position sensor such as, but not limited to, GPS or Glonass, angular rate sensor, shock detector, ultrasonic sensor, tilt sensor, tachometer, ultra-wideband radar, variable reluctance sensor, and velocity receiver.
Imaging, optical and light sensors, such as, but not limited to, CMOS sensor, colorimeter, contact image sensor, electro-optical sensor, infra-red sensor, kinetic inductance detector, LED configured as a light sensor, light-addressable potentiometric sensor, Nichols radiometer, fiber-optic sensors, optical position sensor, thermopile laser sensor, photodetector, photodiode, photomultiplier tubes, phototransistor, photoelectric sensor, photoionization detector, photomultiplier, photoresistor, photoswitch, phototube, scintillometer, Shack-Hartmann, single-photon avalanche diode, superconducting nanowire single-photon detector, transition edge sensor, visible light photon counter, and wavefront sensor.
Pressure sensors, such as, but not limited to, barograph, barometer, boost gauge, bourdon gauge, hot filament ionization gauge, ionization gauge, McLeod gauge, Oscillating U-tube, permanent downhole gauge, piezometer, Pirani gauge, pressure sensor, pressure gauge, tactile sensor, and time pressure gauge.
Force, Density, and Level sensors, such as, but not limited to, bhangmeter, hydrometer, force gauge or force sensor, level sensor, load cell, magnetic level or nuclear density sensor or strain gauge, piezocapacitive pressure sensor, piezoelectric sensor, torque sensor, and viscometer.
Thermal and temperature sensors, such as, but not limited to, bolometer, bimetallic strip, calorimeter, exhaust gas temperature gauge, flame detection/pyrometer, Gardon gauge, Golay cell, heat flux sensor, microbolometer, microwave radiometer, net radiometer, infrared/quartz/resistance thermometer, silicon bandgap temperature sensor, thermistor, and thermocouple.
Proximity and presence sensors, such as, but not limited to, alarm sensor, doppler radar, motion detector, occupancy sensor, proximity sensor, passive infrared sensor, reed switch, stud finder, triangulation sensor, touch switch, and wired glove.

Consistent with the embodiments of the present disclosure, the aforementioned computing device 600 may employ a peripherals sub-module 664 as a subset of the I/O 660. The peripheral sub-module 664 comprises ancillary devices uses to put information into and get information out of the computing device 600. There are 3 categories of devices comprising the peripheral sub-module 664, which exist based on their relationship with the computing device 600, input devices, output devices, and input/output devices. Input devices send at least one of data and instructions to the computing device 600. Input devices can be categorized based on, but not limited to:
Modality of input, such as, but not limited to, mechanical motion, audio, visual, and tactile.
Whether the input is discrete, such as but not limited to, pressing a key, or continuous such as, but not limited to the position of a mouse.
The number of degrees of freedom involved, such as, but not limited to, two-dimensional mice and three-dimensional mice used for Computer-Aided Design (CAD) applications.

Output devices provide output from the computing device 600. Output devices convert electronically generated information into a form that can be presented to humans. Input/output devices perform that perform both input and output functions. It should be understood by a person having ordinary skill in the art that the ensuing are non-limiting embodiments of the aforementioned peripheral sub-module 664:
Input Devices
   Human Interface Devices (HID), such as, but not limited to, pointing device (e.g., mouse, touchpad, joystick, touchscreen, game controller/gamepad, remote, light pen, light gun, infrared remote, jog dial, shuttle, and knob), keyboard, graphics tablet, digital pen, gesture recognition devices, magnetic ink character recognition, Sip-and-Puff (SNP) device, and Language Acquisition Device (LAD).
   High degree of freedom devices, that require up to six degrees of freedom such as, but not limited to, camera gimbals, Cave Automatic Virtual Environment (CAVE), and virtual reality systems.
   Video Input devices are used to digitize images or video from the outside world into the computing device 600. The information can be stored in a multitude of formats depending on the user's requirement. Examples of types of video input devices include, but are not limited to, digital camera, digital camcorder, portable media player, webcam, Microsoft Kinect, image scanner, fingerprint scanner, barcode reader, 3D scanner, laser rangefinder, eye gaze tracker, computed tomography, magnetic resonance imaging, positron emission tomography, medical ultrasonography, TV tuner, and iris scanner.
   Audio input devices are used to capture sound. In some cases, an audio output device can be used as an input device to capture produced sound. Audio input devices allow a user to send audio signals to the computing device 600 for at least one of processing, recording, and carrying out commands. Devices such as microphones allow users to speak to the computer to record a voice message or navigate software. Aside from recording, audio input devices are also used with speech recognition software. Examples of types of audio input devices include, but not limited to microphone, Musical Instrumental Digital Interface (MIDI) devices such as, but not limited to a keyboard, and headset.
   Data AcQuisition (DAQ) devices convert at least one of analog signals and physical parameters to digital values for processing by the computing device 600. Examples of DAQ devices may include, but not limited to, Analog to Digital Converter (ADC), data logger, signal conditioning circuitry, multiplexer, and Time to Digital Converter (TDC).
Output Devices may further comprise, but not be limited to:
   Display devices may convert electrical information into visual form, such as, but not limited to, monitor, TV, projector, and Computer Output Microfilm (COM). Display devices can use a plurality of underlying technologies, such as, but not limited to, Cathode-Ray Tube (CRT), Thin-Film Transistor (TFT), Liquid Crystal Display (LCD), Organic Light-Emitting Diode (OLED), MicroLED, E Ink Display (ePaper) and Refreshable Braille Display (Braille Terminal).
   Printers, such as, but not limited to, inkjet printers, laser printers, 3D printers, solid ink printers, and plotters.
   Audio and Video (AV) devices, such as, but not limited to, speakers, headphones, amplifiers, and lights, which include lamps, strobes, DJ lighting, stage lighting, architectural lighting, special effect lighting, and lasers.
   Other devices such as Digital to Analog Converter (DAC)
Input/Output Devices may further comprise, but not be limited to, touchscreens, networking devices (e.g., devices disclosed in network sub-module 662), data storage devices (non-volatile storage 661), facsimile (FAX), and graphics/sound cards.

All rights, including copyrights in the code included herein, are vested in and the property of the Applicant. The Applicant retains and reserves all rights in the code included herein, and grants permission to reproduce the material only in connection with the reproduction of the granted patent and for no other purpose.

### IV. CLAIMS

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. One or more non-transitory computer readable media comprising instructions which, when executed by one or more hardware processors, causes performance of operations comprising:
receiving, via an electronic interface, Electronic Health Record (EHR) data;
encoding the received EHR data as a fixed-length matrix including a plurality of fixed-length vectors;
providing the matrix to a machine learning model as input to produce a plurality of visit history representations;
for one or more particular visit histories, of the plurality of visit history representations, applying code information associated with the particular visit history;
providing the one or more appended visit histories to one or more masked linear layers to produce a probability matrix, the probability matrix comprising probabilities for each code for each visit; and
producing one or more synthetic EHRs based on repeated sequential generation and sampling from the probability matrix.

2. The computer readable media of claim 1, wherein the received EHR data is formatted as a matrix, such that each column of the matrix represents a unique visit and each row of the matrix represents a unique code.

3. The computer readable media of claim 1, wherein the machine learning model comprises an input module and a plurality of Transformer decoders,
wherein the input module comprises:
a code embedding matrix that maps each visit code to a dense vector representation, and
a positional embedding matrix that captures the relative position of each visit in the sequence;
wherein the input module transforms the input EHR data encoded as the fixed-length matrix to a plurality of initial embeddings, which are provided to the plurality of Transformer decoders.

4. The computer readable media of claim 1, wherein each particular visit history representation, of the plurality of visit history representations comprises collective information from all visits prior to the particular visit;
wherein the appending comprises appending to one or more particular visit history representations, code information associated with the particular visit such that each of the one or more appended visit history representations comprises:
collective information from all visits prior to the particular visit, and
code information associated with the particular visit.

5. The computer readable media of claim 1, wherein the one or more masked linear layers comprises a plurality of masked linear layers including:
a linear layer maintaining the same dimensionality of visit history embedding size and initial fixed-length vector size; and
an upper triangular mask matrix of ones multiplied with the linear layer to create the masked layer;
wherein the plurality of masked linear layer preserves the autoregressive property of the probabilities of the probability matrix.

6. The computer readable media of claim 1, the operations further comprising normalizing the probability matrix using one or more normalization functions, such that the one or more synthetic EHRs are generated based on the repeated generation of and sampling from the normalized probability matrix.

7. A method comprising:
receiving, via an electronic interface, Electronic Health Record (EHR) data;
encoding the received EHR data as a fixed-length matrix including a plurality of fixed-length vectors;
providing the matrix to a machine learning model as input to produce a plurality of visit history representations;
for one or more particular visit histories, of the plurality of visit history representations, applying code information associated with the particular visit history;
providing the one or more appended visit histories to one or more masked linear layers to produce a probability matrix, the probability matrix comprising probabilities for each code for each visit; and
producing one or more synthetic EHRs based on repeated sequential generation and sampling from the probability matrix;
wherein the method is performed by at least one device including a hardware processor.

8. The method of claim 7, wherein the received EHR data is formatted as a matrix, such that each column of the matrix represents a unique visit and each row of the matrix represents a unique code.

9. The method of claim 7, wherein the machine learning model comprises an input module and a plurality of Transformer decoders;
wherein the input module comprises:
a code embedding matrix that maps each visit code to a dense vector representation, and
a positional embedding matrix that captures the relative position of each visit in the sequence;
wherein the input module transforms the input EHR data encoded as the fixed-length matrix to a plurality of initial embeddings, which are provided to the plurality of Transformer decoders.

10. The method of claim 7, wherein each particular visit history representation, of the plurality of visit history representations comprises collective information from all visits prior to the particular visit;
wherein the appending comprises appending to one or more particular visit history representations, code information associated with the particular visit such that each of the one or more appended visit history representations comprises:
collective information from all visits prior to the particular visit, and
code information associated with the particular visit.

11. The method of claim 7, wherein the one or more masked linear layers comprises a plurality of masked linear layers, including:
a linear layer maintaining the same dimensionality (of visit history embedding size and initial fixed-length vector size;
an upper triangular mask matrix of ones multiplied with the linear layer to create the masked layer; and
wherein the plurality of masked linear layers preserve the autoregressive property of the probabilities of the probability matrix.

12. The method of claim 7, further comprising normalizing the probability matrix using one or more normalization functions, such that the one or more synthetic EHRs are generated based on repeated generation of and sampling from the normalized probability matrix.

13. A system comprising:
at least one device including a hardware processor;
the system being configured to perform operations comprising:
receiving, via an electronic interface, Electronic Health Record (EHR) data;
encoding the received EHR data as a fixed-length matrix including a plurality of fixed-length vectors;
providing the matrix to a machine learning model as input to produce a plurality of visit history representations;
for one or more particular visit histories, of the plurality of visit history representations, applying code information associated with the particular visit history;
providing the one or more appended visit histories to one or more masked linear layers to produce a probability matrix, the probability matrix comprising probabilities for each code for each visit; and
producing one or more synthetic EHRs based on repeated sequential generation and sampling from the probability matrix.

14. The system of claim 13, wherein the received EHR data is formatted as a matrix, such that each column of the matrix represents a unique visit and each row of the matrix represents a unique code.

15. The system of claim 13, wherein the machine learning model comprises an input module and a plurality of Transformer decoders;
wherein the input module comprises:
a code embedding matrix that maps each visit code to a dense vector representation, and
a positional embedding matrix that captures the relative position of each visit in the sequence;
wherein the input module transforms the input EHR data encoded as the fixed-length matrix to a plurality of initial embeddings, which are provided to the plurality of Transformer decoders.

16. The system of claim 13, wherein each particular visit history representation, of the plurality of visit history representations comprises collective information from all visits prior to the particular visit;
wherein the appending comprises appending to one or more particular visit history representations, code information associated with the particular visit such that each of the one or more appended visit history representations comprises:
collective information from all visits prior to the particular visit, and
code information associated with the particular visit.

17. The system of claim 13, wherein the one or more masked linear layers comprises a plurality of masked linear layers including:
a linear layer maintaining the same dimensionality (of visit history embedding size and initial fixed-length vector size;
an upper triangular mask matrix of ones multiplied with the linear layer to create the masked layer; and
wherein the plurality of masked linear layers preserve the autoregressive property of the probabilities of the probability matrix.

18. The system of claim 13, further comprising normalizing the probability matrix using one or more normalization functions, such that the one or more synthetic EHRs are generated based on repeated generation of and sampling from the normalized probability matrix.
